(19) United States
(12) Patent Application Publication
(10) Pub. No.: US 2008/0253991 A1
(43) Pub. Date: Oct. 16, 2008

(54) Anti-T Cell and Autoantigen Treatment of Autoimmune Disease

(76) Inventors: Anthony Jevnikar, London (CA); Shengwu Ma, London (CA); William Michael Ainley, Carmel, IN (US); Donald Joseph Merlo, Carmel, IN (US); Sean Michael Russell, Indianapolis, IN (US); Janna M. Armstrong, Indianapolis, IN (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/815,359

(22) PCT Filed: Feb. 6, 2006
(86) PCT No.: PCT/CA06/00144

§ 371 Date: Jun. 23, 2008

Related U.S. Application Data
(60) Provisional application No. 60/649,565, filed on Feb. 4, 2005.

(51) Int. Cl. A61K 45/00; A61K 39/395; A61K 38/00; C12N 15/00; A01H 5/00; A61P 37/00; G01N 33/00; C07K 14/00; C12N 15/11; A61K 38/28

(52) U.S. Cl. 424/85.2; 424/154.1; 424/173.1; 514/12; 514/3; 536/23.5; 435/320.1; 530/351; 800/298; 436/63

(57) ABSTRACT

The invention is directed to a new method for the treatment of new onset Type I diabetes in mammals or for the treatment of pre-Type I diabetic mammals where the method comprises administering (a) anti-T cell therapy to the mammal and administering (b) an autoantigen and optional mucosal antigen composition, wherein (a) and (b) are administered concurrently or sequentially Exemplified is a treatment using a mixture of anti-CD3 antibodies, a glutamic acid decarboxylase (GAD) autoantigen, and an immunoregulatory cytokine Canme GAD sequences are also disclosed.

### FIELD OF THE INVENTION

The invention is directed to the treatment of autoimmune disease in mammals. More specifically the invention is directed to a new method for the treatment and diagnosis of new onset Type I diabetes in mammals.

### BACKGROUND OF THE INVENTION

Type 1 or Insulin Dependent Diabetes Mellitus (IDDM) is an autoimmune disorder mainly of glucose metabolism. Complications of diabetes impair the longevity and quality of life, and include atherosclerotic heart disease, gangrene and stroke, as well as diabetic retinopathy, neuropathy and nephropathy. Symptoms of diabetic neuropathy range from peripheral sensory-deficits (pins and needles/carpal tunnel syndrome) to autonomic neuropathy resulting in bladder and bowel dysfunction. Type 1 diabetes is also responsible for a large proportion of the patients on renal dialysis, the result of diabetes-induced end stage renal disease. The prevalence of myocardial infarction, angina and stroke is 2-3 times greater than in non-diabetics, and the Type 1 diabetic's life span is also shortened.

Type I diabetes actually begins before the clinical manifestations of the disease. It starts with the progressive destruction of beta cells in the pancreas. These cells normally produce insulin. The reduction of insulin response to glucose can be measured during this period, however, ultimately there is massive (>90%) destruction of beta cells in the islets of Langerhans. During the early stages of the disease and beyond, Type I diabetes is characterized by the infiltration of pancreatic islets by macrophages and lymphocytes (helper and killer). The macrophage infiltration is believed to prompt the infiltration of small lymphocytes. While clinicians understand the potential for a drug that can address macrophage involvement early in the disease, no safe therapies have yet been found. Current treatment involves daily frequent injections of insulin. However, this can lead to side effects such as hypoglycemic shock. It is important in the treatment of diabetes to control the blood sugar level and maintain it at a normal level.

Diabetes mellitus is not limited to humans but is also one of the most common endocrinopathies in dogs and cats being associated with considerable morbidity and mortality. Diabetic animals are subject to many of the same problems described in human diabetics, such as increased susceptibility to infection and reduced wound healing. Furthermore, the decreased production insulin as is the case in human Type I diabetes promotes lipolysis and moderate hyperlipidemia leading to atherosclerosis. Some complications of diabetes appear to be specific for animals, in that dogs can develop rapid cataracts leading to blindness, while cats can develop an accelerated neuropathy leading to problems of leg weakness and gait disturbance.

Glycemic control in both humans and animals is critical, however control can often not be achieved except by frequent testing and administration of insulin, which is debilitating for humans and not practical in companion animals. As a result, glycemic control is impaired in diabetic animals even with insulin administration, and there is an accelerated mortality in affected animals (Bennett N. Monitoring techniques for diabetes mellitus in the dog and the cat. Clin Tech Small Anim Pract. 2002 May; 17(2):65-9). Treatment options for animals are currently limited to daily insulin administration as well as islet transplantation which has variable success and requires daily immunosuppression which is costly and in itself has additional toxicities (Salgado D, Reusch C, Spiess B. Diabetic cataracts: different incidence between dogs and cats. Schweiz Arch Tierheilkd. 2000 June; 142:349-53). Long term treatment of diabetic dogs with bovine or porcine insulin can lead to significant reactivity and antibodies which can cross-react with homologous insulin and thus problems in diabetic management. (Davison L J, Ristic J M, Herrtage M E, Ramsey I K, Catchpole B. Anti-insulin antibodies in dogs with naturally occurring diabetes mellitus. Vet Immunol Immunopathol. 2003 Jan. 10; 91(1):53-60).

The immunopathogenesis of diabetic disease in dogs is also very similar to human Type I diabetes, with evidence that injury is mediated primarily by autoreactive lymphocytes. Histopathologic and immunocytochemical studies of pancreas of dogs with spontaneous diabetes mellitus shows extensive pancreatic damage, marked reduction or absence of insulin producing beta cells but with preservation of alpha and delta cells. Also, insulitis lesions are composed of infiltrating mononuclear cells, predominantly lymphocytes but evidence of islet-directed humoral autoimmunity is not detected. (Alejandro R, Feldman E C, Shienvold F L, Mintz D H. Advances in canine diabetes mellitus research: etiopathology and results of islet transplantation. J Am Vet Med Assoc. 1988 Nov. 1; 193:1050-5). T cell responses appear to be directed to autoantigens such as GAD, insulin, and IA-2, again similar to human disease. Considerable speculation exists as the potential for molecular mimicry to have precipitated autoimmune attack to islet beta cells, with exposure to viral infections. T-cell activation by rotavirus and possibly other viruses, and dietary proteins, could trigger or exacerbate beta-cell autoimmunity through molecular mimicry with IA-2 and for rotavirus—GAD. (Honeyman M C, Stone N L, Harrison L C. T-cell epitopes in type 1 diabetes autoantigen tyrosine phosphatase IA-2: potential for mimicry with rotavirus and other environmental agents. Mol Med. 1998 4:231-9). Susceptible animals may be identified by antibody screening for various diabetes autoantigens such as GAD, IA-2 and insulin.

Various therapies have been developed to try to reverse Type I diabetes. Anti-CD3 monoclonal antibodies (mAb) have been utilized to try to suppress immune responses by transient T-cell depletion and antigenic modulation of the CD3/T-cell receptor complex. Anti CD3 mAb applied to adult NOD females (a model of Type I diabetes) within 7 days of the onset of full-blown diabetes produced over 4 months remission of overt disease in most of the mice. The immunosuppression was specific for beta-cell-associated antigens (Chatenoud L, Thervet E, Primo J, Bach J F. Anti-CD3 antibody induces long-term remission of overt autoimmunity in non-obese diabetic mice. Proc Natl Acad Sci USA. 1994 91:123-7). However, there was progressive increase in the incidence of diabetes in treated mice to 4 months and full analysis beyond this time was not shown. There was as well return of insulitis within several weeks of treatment and thus it appears that protection with anti-CD3 antibody alone was not sufficient for disease treatment or reversal. Similarly, in human studies treatment with a non-activating anti CD3 mAb maintained or improved insulin production after one year in 9 of the 12 patients in the treatment group. However, maximal benefit as evidenced by reduced insulin requirements and lower glycated hemoglobin levels was observed at 6 months as compared to 12 months. As well only 2 of the 12 patients had a sustained response (P=0.01) beyond 1 year demonstrating that additional therapy would be required (Herold K C, Hagopian W, Auger J A, Poumian-Ruiz E, Taylor L, Donaldson D, Gitelman S E, Harlan D M, Xu D, Zivin R A, Bluestone J A. Anti-CD3 monoclonal antibody in new-onset type 1 diabetes mellitus. N Engl J. Med. 2002 May 30; 346:1692-8).

Oral immune tolerance is a process by which oral administration of protein antigens can result in diminished peripheral immune responses to a subsequent systemic challenge with the same antigen. The basis for such a regulatory system in mammals is to balance protective mucosal antibody responses to pathogens and attenuating potentially harmful allergic responses to newly encountered food proteins. Oral immune tolerance has, also been viewed as a potential therapeutic strategy for preventing and treating autoimmune diseases such as diabetes when triggering autoantigens such as glutamic acid decarboxylase (GAD) have been identified.

The use of plants as an expression system or “bioreactor” in the production of mammalian antigenic proteins for clinical use offers several unique advantages including high production capacity with near unlimited scale up. Being eukaryotes, plants can also perform post-transcriptional and post-translational modifications required for functional transgenic proteins such as formation of disulfide bonds and folding. As protein isolation costs can eliminate the economic advantage of any production system, an additional practical advantage of transgenic plants for oral tolerance is that plant expression systems can also become effective delivery systems without extensive purification. The composition of plants contains additional compounds, proteins, lectins and other moieties that participate in altering immune responses with the potential to enhance oral tolerance. As well, augmented immune responses to plant produced vaccines may suggest increased stability for plant expressed transgenic proteins to gastrointestinal degradation, and collectively these features make plants an ideal expression and delivery system for oral immune tolerance.

U.S. Pat. No. 6,338,850 discloses a method for oral immune tolerance utilizing a diabetes-associated beta cell autoantigen produced in transgenic plants. Non-obese diabetic (NOD) mice were protected from diabetes when administered such transgenic plant tissue.

In summary, although much progress has been made in the last three decades to understand the mechanisms of Type I diabetes, there is a continual need to develop new and better therapies to treat and possibly reverse the disease in both humans and animals.

### SUMMARY OF THE INVENTION

The present invention is based upon the novel demonstration that the combination of anti-T cell therapy with oral immune tolerance provides a therapy that is more efficacious than either therapy used alone, for the treatment of autoimmune disease and in particular, for the treatment of Type I diabetes.

It is an aspect of the invention to provide a regimen that provides therapeutic benefit to mammals with new onset autoimmune disease such as Type I diabetes and that overcomes some of the disadvantages of currently employed therapies. It is another aspect of the invention to provide improved alternative therapies and regimens for the treatment of Type I diabetes. These and other objectives are accomplished by the present invention which is a novel method for the treatment of new onset Type I diabetes or for the preventative treatment of those at imminent risk for developing Type I diabetes.

The method combines anti-T cell therapy with immune tolerance and is to be for administration to mammals at imminent risk (i.e. pre-diabetic) for developing Type I diabetes or those with new onset Type I diabetes. In embodiments of the invention, the method can be conducted concurrently or sequentially. As a sequential therapy the mammal is first treated with anti-T cell therapy followed by immune tolerance therapy to maintain a disease free state. The method of the invention can be used in combination with any other known diabetic treatments.

The invention is also directed to methods of diagnosis of new onset Type I diabetes in mammals. Such diagnosis comprises the detection of antibodies to for example, glutamic acid decarboxylase (GAD), as a predictor of the development of Type I diabetes. In this aspect, antibodies directed to various forms of GAD may be used in the method. In further aspects, novel gene sequences and novel antibodies directed to novel forms of GAD such as but not limited to GAD65 may be used in the invention. In still further aspects the GAD65 may be canine GAD65 and plant codon optimized genes encoding canine GAD65 as described herein.

According to an aspect of the invention is a treatment regime for Type I diabetes wherein said regime comprises the administration of anti-T cell antibodies and a composition comprising one or more autoantigens with one or more immunoregulatory cytokines to a mammal. The administration may be concurrent or sequential. The treatment regime may be used in conjunction with other known treatments for Type I diabetes. Further, the treatment regime can be used for those mammals that are at imminent risk for developing Type I diabetes.

The method of the invention comprises the use of anti-T cell therapy in conjunction with an autoantigen. However, in further aspects of the invention, the autoantigen portion of the therapy may be used alone or with a mucosal antigen such as an immunoregulatory cytokine.

In all aspects of the invention, the combined use of anti-T cell therapy and autoantigen may be concurrent or sequential. Concurrent therapy is understood by one of skill to involve the administration of anti-T cell therapy with the administration of autoantigen, alternatively, this could mean the administration of anti-T cell therapy together with the administration of autoantigen and then this may also be followed with the further administration of further autoantigen. Concurrent type of administration may be for different time periods as is understood by one of skill in the art and may be followed by further autoantigen therapy for different time periods.

According to an aspect of the present invention is a method for treating Type I diabetes in a mammal or for treating mammals at imminent risk for developing Type I diabetes, the method comprising the combined use of anti-T cell therapy with autoantigen therapy. In aspects, the use may be concurrent or sequential or a combination of both provided at different time intervals.

According to an aspect of the present invention there is a treatment regime for treating Type I diabetes in a mammal or for mammals at imminent risk for developing Type I diabetes, said method comprising:

(a) administering anti-T cell therapy to said mammal; and

(b) administering an effective immunosuppressive dose of a composition comprising at least one autoantigen;

wherein said administering of (a) and (b) is done concurrently or sequentially.

In aspects of the invention, the administration of (b) can be further continued for days and up to several days, weeks, months or years as required.

According to an aspect of the present invention there is a treatment regime for treating Type I diabetes in a mammal or for mammals at imminent risk for developing Type I diabetes, said method comprising:

(a) administering anti-T cell therapy to said mammal; and

(b) administering an effective immunosuppressive dose of a composition comprising at least one autoantigen;

wherein (a) and (b) are administered at the same time; or (a) is administered before (b); or (a) and (b) are administered at the same time and then (b) is further administered for an extended period of time.

According to an aspect of the present invention there is a treatment regime for treating Type I diabetes in a mammal or for mammals at imminent risk for developing Type I diabetes, said method comprising:

(a) administering anti-T cell therapy to said mammal; and

(b) administering an effective immunosuppressive dose of a composition comprising at least one autoantigen and at least one mucosal antigen;

wherein said administering of (a) and (b) is done concurrently or sequentially.

In aspects, (a) and (b) are administered at the same time; or (a) is administered before (b); or (a) and (b) are administered at the same time and then (b) is further administered.

According to a further aspect of the present invention there is provided a method for treating Type I diabetes in a mammal or for mammals at imminent risk for developing Type I diabetes, said method comprising: I

(a) administering an effective immunosuppressive dose of anti-T cell antibodies to said mammal; and

(b) administering an effective immunosuppressive dose of at least one autoantigen and at least one immunoregulatory cytokine;

wherein said administering of (a) and (b) is done concurrently or sequentially.

In aspects, (a) and (b) are administered at the same time; or (a) is administered before (b); or (a) and (b) are administered at the same time and then (b) is further administered.

According to an aspect of the present invention there is provided a method for treating Type I diabetes in a mammal or for mammals at imminent risk for developing Type I diabetes, said method comprising:

(a) administering an effective immunosuppressive dose of anti-T cell antibodies to said mammal; and

(b) administering an effective immunosuppressive dose of a transgenic plant material to said mammal, said transgenic plant material containing at least one autoantigen and an immunoregulatory cytokine;

wherein said administering of (a) and (b) is concurrently done.

In aspects, (b) may be further administered.

According to another aspect of the present invention there is provided a method for treating Type I diabetes in a mammal or for mammals at imminent risk for developing Type I diabetes, said method comprising:

(a) administering an effective immunosuppressive dose of anti-T cell antibodies to said mammal; and

(b) administering an effective immunosuppressive dose of a transgenic plant material to said mammal, said transgenic plant material containing at least one autoantigen and an immunoregulatory cytokine,

wherein said administering of (a) is done first and then administering of (b) is followed.

According to another aspect of the present invention there is provided a method for treating Type I diabetes in a mammal or for mammals at imminent risk for developing Type I diabetes, said method comprising:

(a) administering an effective immunosuppressive dose of anti-CD3 monoclonal antibodies to said mammal; and

(b) administering an effective immunosuppressive dose of a transgenic plant material to said mammal, said transgenic plant material containing a combination of a GAD isoform and IL-4,

wherein said administering of (a) and (b) is concurrently done.

According to another aspect of the present invention there is provided a method for treating Type I diabetes in a mammal, said method comprising:

(a) administering an effective immunosuppressive dose of anti-CD3 monoclonal antibodies to said mammal; and

(b) administering an effective immunosuppressive dose of a transgenic plant material to said mammal, said transgenic plant material containing a combination of a GAD isoform and IL-4,

wherein said administering of (a) is done first and then administering of (b) is followed.

According to another aspect of the invention is a method for the reversal of Type I diabetes in a human or animal, said method comprising;
administering a therapeutically effective amount of anti-CD3 monoclonal antibody to said human or animal; and
administering a therapeutically effective amount of a transgenic plant material containing one or more GAD autoantigens together with IL-4,

wherein said monoclonal antibody is first administered to said human or animal.

According to another aspect of the invention is a method for the reversal of Type I diabetes in a human or animal, said method comprising;
administering a therapeutically effective amount of anti-CDS monoclonal antibody to said human or animal; and
administering a therapeutically effective amount of a transgenic plant material containing one or more GAD autoantigens together with IL-4,

wherein said monoclonal antibody and said transgenic plant material is administered concurrently to said human or animal.

According to yet another aspect of the present invention is a composition comprising a mixture of anti-CD3 antibodies and a preparation that contains at least one autoantigen and an immunoregulatory cytokine.

According to yet another aspect of the present invention is a composition comprising a mixture of anti-CD3 antibodies and a transgenic plant material that contains at least one autoantigen and an immunoregulatory cytokine.

According to another aspect of the present invention is a method for the diagnosis of Type I diabetes in a mammal, the method comprising detecting in a sample from said mammal the presence of anti-GAD antibodies. Such detection being an early indicator of the development or the risk of development of Type I diabetes in the mammal. In aspects of the invention, the method may comprise the use of canine GAD65.

According to a further aspect of the present invention are novel GAD65 sequences, such sequences may be used for plant transformation. In aspects, the sequence is a canine GAD65 sequence of SEQ ID NO:4. In further aspects, the sequence is an optimized GAD65 sequence of SEQ ID NO:5.

According to still further aspects of the present invention are novel IL4 sequences, such sequences may be used for plant transformation. In aspects, the sequence is a canine IL4 sequence optimized for plant expression and is represented by SEQ ID NO:2 or SEQ ID NO:7.

In other aspects of the invention are vectors for the transformation of plant cells. In aspects, these vectors contain sequences selected from the group consisting of SEQ ID NO:1, SEQ ID NO:3 AND SEQ ID NO. 6.

According to still a further aspect of the present invention is the use of a composition comprising anti-T cell antibodies, autoantigen and optional mucosal antigen in the manufacture of a medicament for the treatment of Type I diabetes in a mammal.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE FIGURES

The present invention will become more fully understood from the description given herein, and from the accompanying drawings, which are given by way of illustration only and do not limit the intended scope of the invention.

FIG. 1 shows the effect of control plant feeding versus GAD/IL-4 plant feeding on blood glucose levels in diabetic female NOD mice.

FIG. 2 shows a Kaplan Meier Survival analysis demonstrating the time to hyperglycemia for the diabetic female NOD mice of FIG. I.

FIG. 3 shows blood glucose levels post feeding at baseline, day 40 and day 60 after anti CD3 therapy.

FIG. 4 shows the level of anti-GAD IgG1 in GAD/IL-4 fed mice compared to controls.

FIG. 5 shows the delayed mean time to diabetes for GAD/IL-4 fed mice compared to controls.

FIG. 6 shows the levels of serum anti-GAD65 antibodies in healthy and newly diagnosed diabetic dogs by anti-GAD ELISA.

FIG. 7 shows Western blot analysis of canine GAD65 protein expression in transgenic tobacco plants. Total protein extracts (40 μg/lane) from transgenic tobacco leaf tissue were fractionated by sodium dodecylsulphate-polyacrylamide gel electrophoresis (SDS-PAGE), blotted on to polyvinylidene difluoride (PVDF) membrane and probed with an anti-GAD antibody. Lanes 1 to 5, independent canine GAD65 transgenic tobacco lines; WT, wild-type tobacco; GAD67, used as a positive control. Numbers on the left indicate the positions of protein size markers.

FIG. 8A shows that plant rcIL-4 stimulates the ³H-thymidine incorporation by TF-1 cells in a dose-dependent manner. Pre-incubation of plant rcIL-4 with anti-canine IL-4 antibody (Ab) reduces its ability to stimulate TF-1 cells in proliferation. FIG. 8B shows the incorporation of ³H-thymidine by TF-1 cells in response to stimulation with a standard commercial source of rcIL-4.

FIGS. 9A-9N are various plasmid constructs used for cell transformations. FIG. 9A pDAB771; FIG. 9B pDAB773; FIG. 9C pDAB2407; FIG. 9D pDAB2457; FIG. 9E pDAB2455; FIG. 9F pDAB2456; FIG. 9G pDAB3736; FIG. 9H pDAB3741; FIG. 9I pDAB3731; FIG. 9J pDAB3748; FIG. 9K pDAB2453; FIG. 9L pDAB4005; FIG. 9M pDAB2451; and FIG. 9N pDAB8504.

FIG. 10 shows western blots of IL-4 expression in transgenic tobacco. Calli were extracted in SDS gel loading solution and heated at 95° C. Gels and westerns were performed as described in the examples section.

FIG. 11 shows western blots of IL-4 expression in transgenic rice. Calli were extracted in SDS gel loading solution and heated at 95° C. Gels and westerns were performed as described in the examples section.

FIG. 12 shows western analysis of the cGAD65 samples expressed in NT-1 calli. Calli were extracted in SDS gel loading solution and heated at 95° C. The arrow indicates the recombinant standard rhGAD65. Lane 1: molecular weight markers; lane 2: rhGAD65 standard; lane 3: non-transgenic callus; lanes 4-13: independent cGAD65 transgenic events.

FIG. 13 shows the purification of IL-4. cIL-4 produced in transgenic tobacco callus was purified as described above. The chromatograph of the Hi-Trap Nickel column is shown, with the fractions retained for further purification. SDS-PAGE analysis of the fractions eluted from the Superose 6 column identified a major protein band (arrow) that corresponded to cIL-4 as determined by western blot and MALDI-TOF analysis.

### DETAILED DESCRIPTION OF THE PREFERRED EMBODIMENTS

The invention is a new treatment method/regime for autoimmune disease and in particular, for the treatment of Type 1 diabetes in mammals. The method is a combination treatment whereby anti-T cell therapy is combined with immune tolerance in a mammal. The combination treatment may be done concurrently or sequentially. The combination of anti-T cell therapy with immune tolerance provides a therapy that is more efficacious than either therapy alone, particularly for the treatment of Type I diabetes. Using the method of the present invention, it was demonstrated that there was no reverting to diabetes for an extensive period of time demonstrating that the method is an effective long term treatment and can in fact reverse diabetes.

The method of the invention is useful for the treatment of new onset Type I diabetes in a mammal. The method of the invention is also useful for the treatment of mammals at imminent risk for developing Type I diabetes which includes mammals with impaired islet cell function due to autoimmunity but not requiring insulin therapy. In this aspect, such mammals are considered pre-diabetic. The method can be used to reverse Type I diabetes in mammals.

It is understood by those of skill in the art that the method of the invention can be used in conjunction with other known treatments suitable for Type I diabetes.

### DEFINITIONS

As referred to herein, Type 1 diabetes is generally understood to be an auto-immune disease and is also referred to in the general literature as: type 1 DM, insulin-dependent diabetes, IDD, insulin-dependent diabetes mellitus, IDDM, childhood diabetes, childhood diabetes mellitus, childhood-onset diabetes, childhood-onset diabetes mellitus, diabetes in childhood, diabetes mellitus in childhood, juvenile-onset diabetes, juvenile-onset diabetes mellitus, and autoimmune diabetes mellitus.

As referred to herein, autoantigens are native proteins or peptides that, in some individuals, are immune response-provoking. When autoantigens are administered to such individuals, the autoantigens induce tolerance or suppress the immune response of the mammal to the protein or peptide.

As referred to herein, mucosal adjuvants are immunological agents which work through or at the mucosal surface, or at lymphoid structures associated with the gut and increase the antigenic response. Mucosal adjuvants as disclosed herein may be immunoregulatory cytokines that are any of several regulatory proteins, such as cytokines and interleukins that are released by cells of the immune system and act as intercellular mediators in the generation of an, immune response. Cytokines may include those released by lymphocytes, other immune cells or parenchymal cells upon activation which can modify, attenuate or eliminate harmful autoimmune responses directed to a specific antigen or antigens.

As referred to herein, GAD (glutamic acid decarboxylase) encompasses different GAD isoforms as well as GAD polypeptides that contain one or more GAD epitopes recognized by autoantibodies.

As referred to herein, the term “transgenic plant material” as used herein is any type of transgenic plant material that contains the autoantigen and mucosal adjuvant as expressed by the plant. The plant material may include but not be limited to plant tissue, plant part (i.e. leaves, tubers, stems etc.), plant cell cultures including but not limited to plant suspension cultures and plant callus cultures, plant extracts, plant slurries and combinations thereof. The plant material can be provided “raw” or processed in some manner so long as it contains the transgenic protein of interest. Methods for processing plant material that are consistent with use in the present invention may be found in WO2002083072, WO2004098530, and WO2004098533 (the disclosures of which are herein incorporated by reference in their entirety).

As referred to herein, “mammal” includes any warm-blooded animal with mammary glands. A preferred group of mammals is the group consisting of humans and companion animals. In aspects, this group consists of humans, dogs, cats and horses. In preferred aspects, this group consists of dogs and cats and in most preferred aspects this group is humans.

### Anti-T Cell Therapy

In the method, the anti-T cell therapy is administered to the mammal to cause T cell depletion. The anti-T cell therapy may be any effective immunosuppressant agent that targets T cells. In aspects of the invention this may include but not be limited to monoclonal antibodies and polyclonal antibodies that target surface antigens on T cells or alternatively other agents such as cyclosporine, methotrexate and azathioprine.

In aspects of the invention suitable antibodies may be selected from but not be limited to anti CD3, anti CD2, anti CD4, anti CD7, anti CD8, anti CD25, anti CD28, alpha 4 beta 1 integrin, alpha 4 beta 7 integrin and other T cell surface antigens as is well understood by those of skill in the art. The selected T cell depletion agent such as an antibody as herein described would then be administered to the mammal in need thereof. Treatment with the antibody would be done for up to about 10 days. This time period can be varied as is understood by one of skill in the art. In aspects, this time period can be about 5 to 7 days.

The T cell depletion agent may be administered at dosages of about 10 μg/kg to about 100 μg/kg body weight intravenously. This may include any range thereinbetween such as but not limited to 10 μg/kg to about 20 μg/kg body weight; 20 μg/kg to about 30 μg/kg body weight; 30 μg/kg to about 40 μg/kg body weight; 40 μg/kg to about 50 μg/kg body weight; 50 μg/kg to about 60 μg/kg body weight; 60 μg/kg to about 70 μg/kg body weight; 70 μg/kg to about 80 μg/kg body weight; 80 μg/kg to about 90 μg/kg body weight; and 90 μg/kg to about 100 μg/kg body weight. It is also understood by one of skill in the art that the dose range may differ from the described range and thus may not be limited to this range depending on the species of mammal and should be a dosage that essentially eliminates circulating T cells, as measured in peripheral blood. Determination of the suitable dose of T cell depletion agent may be accomplished by detection of monoclonal antibodies on the surface of circulating T cells. Responsiveness to the antibody treatment may be confirmed by measurement of blood sugar levels whereby mammals exhibiting levels under control and within normal ranges are then considered to be responsive to the treatment.

In one representative but non-limiting embodiment of the invention, anti-T cell therapy is effectively accomplished by the administration of anti-CD3 monoclonal antibodies for about 5 to about 7 days.

### Immune Tolerance

The method of the invention also incorporates immune tolerance. Immune tolerance is achieved by administering to the mammal one or more autoantigens and, optionally, one or more mucosal adjuvants. The autoantigen and optional mucosal adjuvant may be co-administered when a mucosal adjuvant is used. Immune tolerance can be administered concomitantly with the anti-T cell therapy or after the anti-T cell therapy is completed. Furthermore, immune tolerance treatment may be administered after concurrent first administration of the combined anti-T cell therapy and oral tolerance. In other words, the autoantigen and optional mucosal antigen can be administered for a time period as required. Thus the autoantigen may be administered for an extended period of time to the mammal in need of the treatment that is well beyond the time of anti-T cell therapy administration. In some aspects this may be the lifespan of the mammal if continued administration is required.

If administered after completion of the anti-T cell therapy, oral immune tolerance can be delayed for up to about 4 weeks. In aspects of the invention, the immune tolerance is mucosal immune tolerance whereby the autoantigen and mucosal adjuvant are co-administered via a mucosal surface. In these aspects of the invention a preferred mucosal immune tolerance is induced orally.

The autoantigen selected is the trigger antigen responsive for the autoimmune disease. In the case of Type I diabetes, the autoantigen is selected from the group consisting of species specific or species non-specific GAD (glutamic acid decarboxylase) isoforms and GAD polypeptides. GAD isoforms are known to those of skill in the art and may include but not be limited to GAD65 and GAD67. Still other autoantigens may be selected from the group consisting of insulin and beta cell proteins capable of eliciting harmful autoimmune responses. The amount of autoantigen that may be used for administration was found to be about 7-8 μg/25 gm mouse. Thus the amount of autoantigen for use in the method of the invention is about up to 300 μg/kg for a mammal and any ranges thereinbetween. Thus suitable amounts may include but not be limited to about 1 μg/kg to 1000 μg/kg; 10 μg/kg to 800 μg/kg; about 50 μg/kg to 700 μg/kg; about 100 μg/kg to 500 μg/kg; and about 200 μg/kg to 400 μg/kg. Dosage amounts for a particular mammal may be varied as is understood by one of skill in the art.

It is also understood by those of skill in the art that the GAD sequences used in the present invention may be of any species such as but not limited to human, feline and canine sequences. The human sequence is disclosed in Bu et al., 1992. Two human glutamate decarboxylases, 65-kDa GAD and 67-dDa GAD are each encoded by a single gene. Proc Natl Acad Sci USA 89:2115-2119 (the disclosure of which is incorporated herein by reference in its entirety). The feline GAD sequence is disclosed in Kobayashi et al., 1987. Glutamic acid decarboxylase cDNA: nucleotide sequence encoding an enzymatically active fusion protein. J. Neurosci. 7:2768-2772 (the disclosure of which is incorporated herein by reference in its entirety). Canine sequences for use in the invention may include those of native canine GAD65 (SEQ ID NO.4) and canine GAD65 (SEQ ID NO.5) having a polyhistidine purification tag which was codon optimized for plant expression.

The GAD peptide sequences for use in the invention may be obtained by chemical synthesis using automated instruments or alternatively by expression from nucleic acid sequences which are capable of directing synthesis of the peptide using recombinant DNA techniques well known to one skilled in the art. GAD peptides of the invention may be prepared by chemical synthesis using techniques well known in the chemistry of proteins such as solid phase synthesis (Merrifield, J. Am. Chem. Assoc. 85:2149-2154 (1964)) or synthesis in homogenous solution (Houbenweyl, Methods of Organic Chemistry (1987), (Ed. E. Wansch) Vol. 15, pts. I and II, Thieme, Stuttgart). Techniques for production of proteins by recombinant expression are well known to those in the art and are described, for example, in Sambrook et al. (1989) or latest edition thereof.

Also encompassed by the canine GAD nucleic acid sequences of the invention are complementary as well as anti-complementary sequences to a sequence encoding and equivalent sequence variants thereof. One skilled in the art would readily be able to determine such complementary or anti-complementary nucleic acid sequences. Also as part of the invention are nucleic acid sequences which hybridize to one of the aforementioned nucleic acid molecules under stringent conditions. “Stringent conditions” as used herein refers to parameters with which the art is familiar and such parameters are discussed, for example, in the latest editions of Molecular Cloning: A Laboratory Manual, J. Sambrook, et al., eds., Cold Spring Harbor Laboratory Press, Cold Spring Harbor, N.Y., or Current Protocols in Molecular Biology, F. M. Ausubel, et al., eds., John Wiley & Sons Inc., New York. One skilled in the art would be able to identify homologues of nucleic acids encoding the BCSP peptides of the invention. Cells and libraries are screened for expression of such molecules which then are routinely isolated, followed by isolation of the pertinent nucleic acid molecule and sequencing.

It is noted that the nucleic acid molecules described herein may also encompass degenerate nucleic acids. Due to degeneracy in the genetic code, variations in the DNA sequence will result in translation of identical peptides. It is thus understood that numerous choices of nucleotides may be made that will lead to a sequence capable of directing production of the peptides or functional analogues thereof of the present invention. As a result, degenerative nucleotide substitutions are included in the scope of the invention.

Allowing for the degeneracy of the genetic code as well as conserved and semi-conserved substitutions, sequences which have between about 40% and about 80%; or more preferably, between about 80% and about 90%; or even more preferably, between about 90% and about 99%; of nucleotides which are identical to the nucleotides of SEQ ID NO:2, 4, 5 and 7 will be sequences which are “essentially as set forth in SEQ ID NO:2, 4, 5 and 7”. Sequences which are essentially the same as those set forth in SEQ ID NO:2-4, 5 and 7 may also be functionally defined as sequences which are capable of hybridizing to a nucleic acid segment containing the complement of SEQ ID NO:2, 4, 5 and 7 under standard or less stringent hybridizing conditions. Suitable standard hybridization conditions will be well known to those of skill in the art.

As would be understood by one of skill in the art, nucleic acid molecules of the present invention may encompass single and double stranded forms, plasmid(s), viral nucleic acid(s), plasmid(s) bacterial DNA, naked/free DNA and RNA. A viral nucleic acid comprising a nucleic acid sequence encoding for at least one peptide of the invention may be referred to as a viral vector.

The invention also encompasses expression vectors comprising the nucleic acid sequences of the invention of SEQ ID NO. 2, 4, 5 and 7 and functional analogues thereof within expression vectors. Any expression vector that is capable of carrying and expressing the nucleic acid sequences encoding for the peptides of the invention and functional analogues thereof in prokaryotic or eukaryotic host cells may be used, including recombinant viruses such as poxvirus, adenovirus, alphavirus and lentivirus. The invention also encompasses host cells transformed, transfected or infected with such vectors to express the peptides or functional analogues of the invention. As such, host cells encompass any potential cell into which a nucleic acid of the present invention may be introduced and/or transfected.

The optional mucosal adjuvant for use in conjunction with the autoantigen may be selected from an immunoregulatory cytokine such as but not limited to the interleukins: IL-1, IL-2, IL-3, IL-4, IL-5, IL-6, IL-7, IL-8, IL-9, IL-10, IL-12, IL-13, IL-15 and IL-18. Any cytokine that is released by lymphocytes, other immune cells or parenchymal cells upon activation which can modify, attenuate or eliminate harmful autoimmune responses directed to a specific antigen or antigens is suitable for use in the present invention as is understood by one of skill in the art. In aspects of the invention, the cytokine used is IL4 of which may be of various species origin such as for example but not limited to human (as described in Yokota et al., 1986. Proc Natl Acad. Sci USA. 83:5894-5898, the disclosure of which is incorporated by reference herein in its entirety) and canine (as described in Lee et al., 1986. Proc Nati Acad Sci USA. 83:2061-2065, the disclosure of which is incorporated herein by reference in its entirety). In these aspects a suitable canine IL-4 sequence is represented by SEQ ID NO.2 and SEQ ID NO. 7 which are optimized for plant expression. As with GAD described above, IL-4 sequences as disclosed herein may encompass various forms and be incorporated into various constructs for use in cell transfection. Suitable amounts of cytokine for use in the invention has been demonstrated to be about 1-2 μg/25 gm in the mouse. Thus in mammals suitable amounts for use in the methods are up to about 500 μg/kg and any range thereinbetween such as for example about 0.5 μg/kg to about 500 μg/kg; about 1.0 μg/kg to about 250 μg/kg; and about 10.0 μg/kg to about 100 μg/kg. One of skill in the art would clearly understand amounts of suitable dosages for use in the present invention.

The autoantigen and optional mucosal adjuvant may be administered as a composition. The compositions may be administered alone or in combination with at least one other agent, such as stabilizing compound, which may be administered in any, biocompatible pharmaceutical carrier, including, but not limited to, saline, buffered saline, dextrose, and water. The compositions may be administered to the subject alone, or in combination with other agents or drugs.

The pharmaceutical compositions encompassed by the invention may be administered by any number of routes. Pharmaceutical compositions for oral and mucosal administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Pharmaceutical preparations which can be used orally include capsules made of gelatin, as well as soft, sealed capsules made of gelatin and a coating, such as glycerol or sorbitol. Such capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral (intravenous and intramuscular) administration may be formulated m aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

The pharmaceutical composition may be provided in biodegradable microspheres as is disclosed in Sinha et al., Journal of Controlled Release 90 (2003) 261-280 (the disclosure of which is incorporated herein by reference).

In one embodiment of the invention, immune tolerance is achieved via a method of oral immune tolerance where the autoantigen and the mucosal adjuvant are administered within an edible plant material and as such are produced by a transgenic plant that contains the required sequences that are expressed by the plant to produce the proteins in the plant. The expression of GAD autoantigens in plants is described in U.S. Pat. No. 6,338,850 (the disclosure of which is incorporated herein by reference in its entirety). Autoantigens and mucosal adjuvants can be successfully produced in transgenic plants as is disclosed in Ma S, Huang Y, Yin Z, Menassa R, Brandle J E, Jevnikar A M Induction of oral tolerance to prevent diabetes with transgenic plants requires-glutamic acid decarboxylase (GAD) and IL-4. Proc Natl Acad Sci USA. 2004 Apr. 13; 101(15):5680-5 and in Ma S W, Zhao D L, Yin Z Q, Mukherjee R, Singh B, Qin H Y, Stiller C R, Jevnikar A M. Transgenic plants expressing autoantigens fed to mice to induce oral immune tolerance. Nat Med. 1997 July; 3(7):793-6 (the disclosures of which are incorporated herein by reference in their entirety).

Briefly, to construct a transgenic plant expressing an autoantigen, a cDNA coding for a selected autoantigen such as for example human GAD may be inserted into an expression vector and used to create transgenic plants by means of Agrobacterium-mediated transfection, as described herein in a representative but non-limiting example (Example A). In this example, a potato plant is used as transgenic starch tubers provide a very inexpensive source of biomass for heterologous protein production. Transgenic plants expressing the desired antigen may be identified by examination of plant extracts by Western blotting, by conventional techniques, expressed antigen being detected by means of an appropriate specific antibody. Where the antigen to which tolerance is desired has a heterodimeric structure, one may either transform plant tissue sequentially with two vectors, each carrying the DNA for an individual protein chain and a different selection in marker gene, so that the plant produces the mature antigen, or one may introduce the DNA for each chain into separate plants and breed these, by cross-pollination of “single chain” plants by standard techniques to give hybrids producing the mature antigen.

The transgenic plant material containing the expressed antigen may be administered orally or enterally to the subject in an effective dose. The particular selection of plant for transgenic manipulation may be edible or non-edible. If a non-edible plant species is used for production of mammalian antigens, the antigens may be extracted from the plant tissue and purified as required by standard methods before oral or enteral administration.

The transgenic plant material can be administered to the mammal in need of as required. In order to produce oral tolerance in a subject to a particular mammalian antigen, transgenic plant tissue containing the expressed antigen may be administered orally or enterally to the subject in an effective dose as described herein supra. Alternatively, if a non-edible transgenic plant is used for production of mammalian antigens, the antigens may be extracted from the plant tissue and purified as required by standard methods before oral or enteral administration. This can include a single administration, multiple administration over time or continued lifetime use. Representative suitable plants for use in the invention may include but are not limited to potato, tomato, alfalfa, canola, rice, tobacco, maize, algae, safflower, moss and bryophyte.

The amount of expressed autoantigen and mucosal adjuvant when used in combination for administration to provide a therapeutic effect is provided on a weight basis and may range in combination from up to about 1 mg/kg to up to about 1000 mg/kg or more along with the plant matrix. In aspects the amount is from up to about 1 mg/kg to up to about 100 mg/kg. It is understood by those of skill in the art that the amount of expressed autoantigen and mucosal adjuvant may vary and may be selected from any sub-range of the 1 mg/kg to about 1000 mg/kg range, such as for example but not limited to; 1 mg/kg-500 mg/kg; 1 mg/kg-250 mg/kg; 1 mg/kg-200 mg/kg; 1 mg/kg-150 mg/kg; 1 mg/kg-75 mg/kg; 1 mg/kg-50 mg/kg; and 1 mg/kg-25 mg/kg and any sub-ranges of any of these ranges. Again, it is also possible that the amount may be greater than 1000 mg/kg and in some aspects less than 1 mg/kg. The amount used in the invention may be species specific as is understood by one of skill in the art.

Various methods are available to identify autoantigen and cytokine production in plants such as with the use of cross reactive monoclonal human and other species antibodies which can be applied to flow cytometric, Western blot analyses and ELISA studies (Pedersen L G, Castelruiz Y, Jacobsen S, Aasted B. Identification of monoclonal antibodies that cross-react with cytokines from different animal species. Vet Immunol Immunopathol. 2002 88:111-22).

The invention also encompasses therapeutic compositions comprising a mixture of T-cell immunosuppressant agent, at least one autoantigen and optionally at least one mucosal adjuvant. In a non-limiting aspect of the invention this may be represented by a composition comprising a GAD isoform, anti-CD3 monoclonal antibody and IL-4. In a further non-limiting aspect of the invention this may be represented by a composition comprising GAD65 and/or GAD67, anti-CD3 monoclonal antibody and IL-4 and/or IL-10. Such a composition may be formulated as herein described for oral or parenteral administration.

It is also understood by one of skill in the art that the method of the invention in its various embodiments can be practiced in conjunction with other treatments currently known and used for the treatment of Type I diabetes. Such treatments may include but not be limited to insulin therapy.

The invention also encompasses the use of anti-GAD65 antibodies for the early detection of Type I diabetes. In this aspect, mammalian sera may be assayed for the presence of anti-GAD65 antibodies which is a predictor of diabetic risk or a diagnostic of Type I diabetes in early stages. In further aspects, such methods may be used in non diabetic identified animals for early detection of diabetes assessment of risk. In these aspects, various types of anti-GAD65 antibodies may be used including novel canine antibodies.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example A

### The Effect of Control Plant Feeding Versus GAD/IL-4 Plant Feeding on Blood Glucose Levels in Diabetic Female NOD Mice

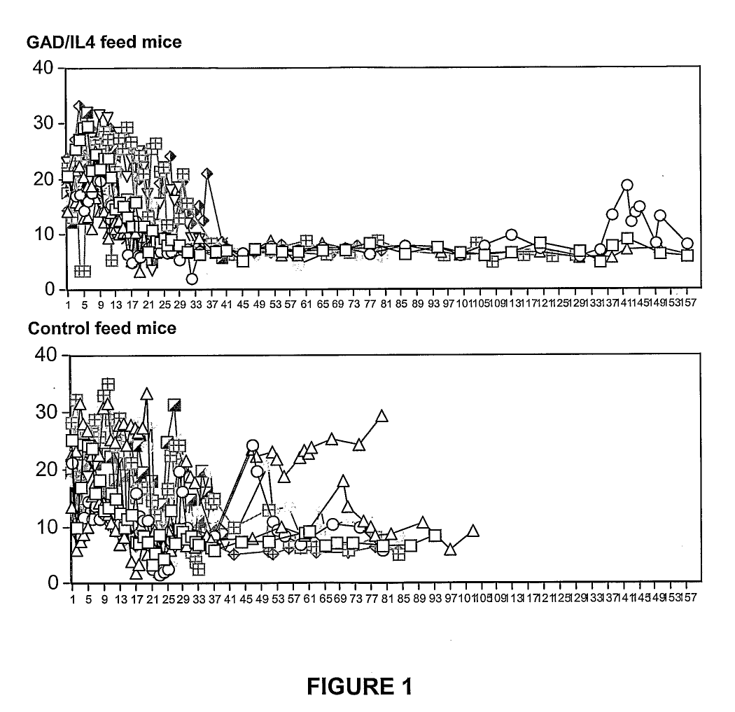

Diabetic female NOD mice were identified by positive urine glucose followed by blood testing. Mice were selected for antibody therapy if blood glucose was greater than 16 mmol/l on 2 consecutive days. Mice were treated with daily insulin to keep blood glucose levels less than approx. 14 mmol/l. Some mice required twice daily insulin. Mice were stabilized and given anti CD3 mAb (5 μg) IV by tail vein injection for 6 days (d1-6). Mice that failed to achieve (on 2 consecutive days), blood sugars less than 20 mmol/l by 3 weeks were terminated. More than 85% of mice achieved this. At 2 to 3 weeks following anti CD3 mAb treatment, mice were given either control plant chow (empty vector, LN tobacco) or GAD-IL4 plant chow and followed daily for blood sugars. Insulin was stopped by 3 weeks in all mice. 10 mice were assigned to each treatment group, and expressed values are mmol/l glucose vs day of experiment (FIG. 1). Day 1 indicates the start of anti CD3 therapy. One GAD/IL4 mouse had late transient hyperglycemia. One GAD/IL-4 mouse was lost to technical problem (insulin OD with hypoglycemia).

Kaplan Meier Survival Analysis Demonstrating the Time to Hyperglycemia for the Diabetic Female NOD Mice.

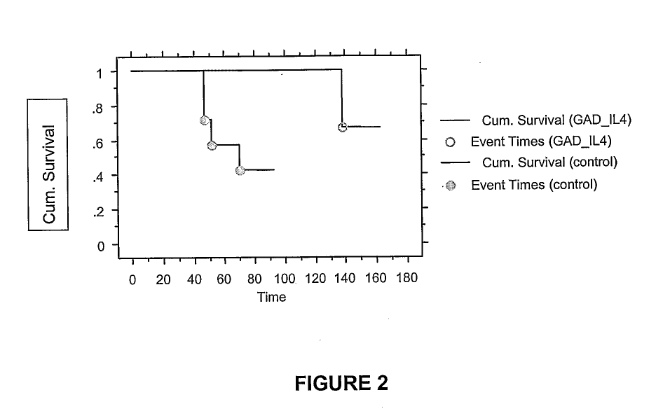

Diabetic female NOD mice of FIG. 1 were followed by blood testing and were given either control plant chow (empty vector, LN tobacco) or GAD-IL4 plant chow One GAD/IL-4 mouse was lost to technical problem (insulin OD with hypoglycemia) and was not considered a treatment failure (censored). Survival was defined as time to hyperglycemia (glucose>12 mmol/l on two consecutive days), at 40 days or beyond: 40 days was selected as mice had stabilized early fluctuations in blood glucose levels (FIG. 1) noted in most mice. The analysis is shown in FIG. 2.

Blood Glucose Levels Post Feeding

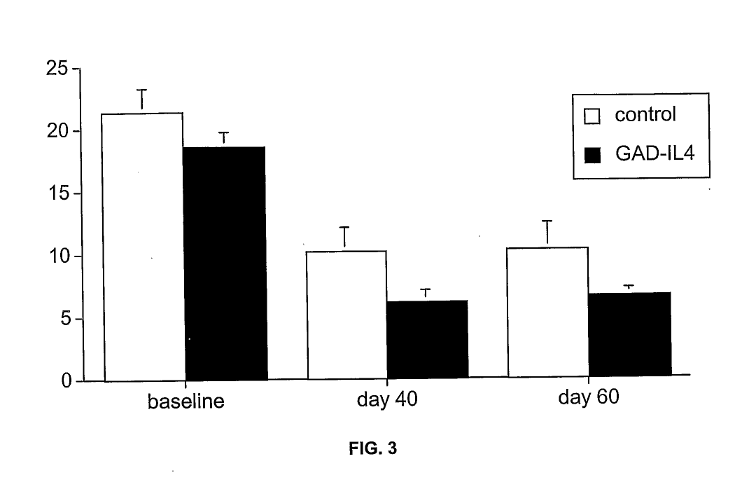

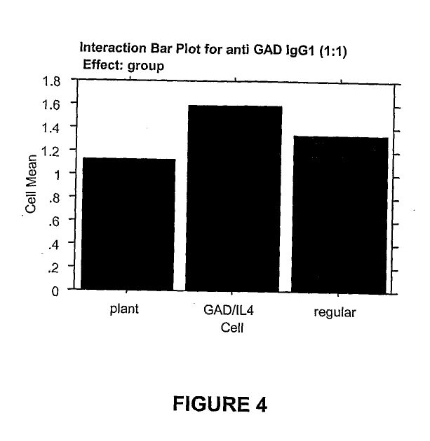

Mice were analyzed for blood glucose levels at baseline, day 40 and day 60 so that direct comparisons could be made for a specific interval after anti CD3 therapy (FIG. 3). Day 40 levels are different (p=0.03) but differences also noted on day 60 did not reach significance (p=0.1). Note that mice at baseline were treated with insulin but at start of treatment with anti CD3 antibody. There is no difference between control and GAD-IL4 regardless at baseline. Days were selected at day 40 and 60 as there sufficient mice to assess both groups (n=10 at baseline and day 40, n=6-7 at day 60).

Production of Transgenic Plants Expressing Autoantigen

Human GAD Expression in Potatoes: Two cDNA clones encoding portions of human GAD were used, representing either the 5′ sequence or the 3′ sequence. The two clones had an overlap sequence of no more than 70 nt. The complete human GAD sequence was made by a series of DNA manipulations. The N-terminal end of human GAD was modified by PCR to include the incorporation of a NcoI (CCATGG) restriction site as part of a translation initiation site. The native 3′ nontranslated sequence, including poly A tail, was completely removed. The modified human GAD sequence was cloned into plasmid vector pTRL-GUS to replace the GUS gene. The plasmid pTRL GU3 is composed of CaMV 35S promoter with double enhancer sequence (Ehn-35S) linked to 5′ untranslated TEV leader sequence, GUS gene and NOS-terminator. The new expression cassette, consisting of 5′-Ehn35S-TEV5′ untranslated leader human GAD-NOS terminator was excised with HindIII and inserted into the binary vector pBIN19. The final construct, designated pSM215, was transferred into agrobacterium and potato transformation was carried out by the leaf disc method (Horsch et al., (1985), Science, vol. 227, pp. 1229-1231). Regeneration of transformed leaf disk into new plants was according to Horsch et al. Primary screening of transformants was based on callus formation on MSO media supplemented with kanamycin.

A more complete experimental protocol for autoantigen expression in plants is provided in Ma S, Huang Y, Yin Z, Menassa R, Brandle J E, Jevnikar A M Induction of oral tolerance to prevent diabetes with transgenic plants requires glutamic acid decarboxylase (GAD) and IL-4. Proc Natl Acad Sci USA. 2004 Apr. 13; 101(15):5680-5 and in Ma S W, Zhao D L, Yin Z Q, Mukherjee R, Singh B, Qin H Y, Stiller C R, Jevnikar A M (the disclosures of which are incorporated herein by reference in their entirety). One of skill in the art could follow these teachings to practice the present invention.

### Example B

### Sequential Anti-CD3 and GAD/IL-4 Feeding in the NOD Mouse Model Showing Effect of Anti-CD3 and Sequential GAD/IL-4 Feeding on Th1/Th2 T Cell Subsets

Female NOD mice were allowed to spontaneously develop diabetes and maintained glycemic control with insulin. Mice were treated daily with anti-CD3 mAb (5 μg) for 6 days and insulin was discontinued on reaching euglycemia. Mice were fed with oral GAD/IL-4, plant control diet or regular mouse chow for 4 weeks (n=3 per group). Mice were euthanized and serum was tested for anti-GAD IgG1 as measure of Th2 activity. GAD/IL-4 mice had the highest level of anti-GAD IgG1 antibodies (p=0.1) suggesting the benefit of anti-CD3 mAb with sequential GAD/IL-4 is related to Th2 skewing of T helper cell subsets.

Means Table for anti GAD IgG1 (1:1)
Effect: group

| | Count | Mean | Std. Dev. | Std. Err. |
|---|---|---|---|---|
| plant | 3 | 1.131 | .273 | .157 |
| GAD/IL4 | 3 | 1.587 | .395 | .228 |
| regular | 3 | 1.336 | .201 | .116 |

### Effect of Anti-CD3 and Sequential GAD/IL-4 on Ability to Prevent Adoptive Transfer of Diabetes

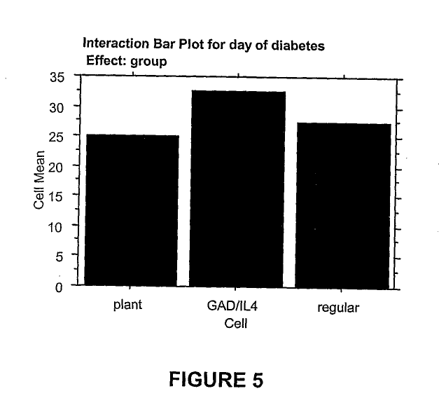

Female NOD mice were allowed to spontaneously develop diabetes and maintained glycemic control with insulin. Mice were treated daily with anti-CD3 mAb (5 ug) for 6 days and insulin was discontinued on reaching euglycemia. Mice were fed with oral GAD/IL-4, plant control diet or regular mouse chow for 4 weeks (n=3 per group) and are the same as mice presented in FIG. 1. Mice were euthanized and spleen cells were isolated from mice from each group were mixed with diabetogenic spleen cells taken from recently diabetic NOD mice. Cells from fed mice (1×10⁷) were mixed with diabetogenic spleen cells ((1×10⁷) and were injected IV into NOD-scid mice. Recipient mice were followed for diabetes by urine testing and confirmed by serum testing (>14 mmol/l for 2 days) and then terminated. Mice receiving oral GAD/IL-4 had delayed me and time to diabetes (p=0.1) compared to plant controls suggesting the benefit of anti-CD3 mAb with sequential GAD/IL-4 is related to induction of regulatory Th2 helper cell subsets (FIG. 5).

Means Table for day of diabetes
Effect: group

| | Count | Mean | Std. Dev. | Std. Err. |
|---|---|---|---|---|
| plant | 7 | 25.000 | 4.320 | 1.633 |
| GAD/IL4 | 6 | 32.500 | 10.691 | 4.365 |
| regular | 6 | 27.500 | 7.369 | 3.008 |

### The Relevance of Elevated GAD65 Antibodies to Diabetes of Non Human or Mouse Mammals.

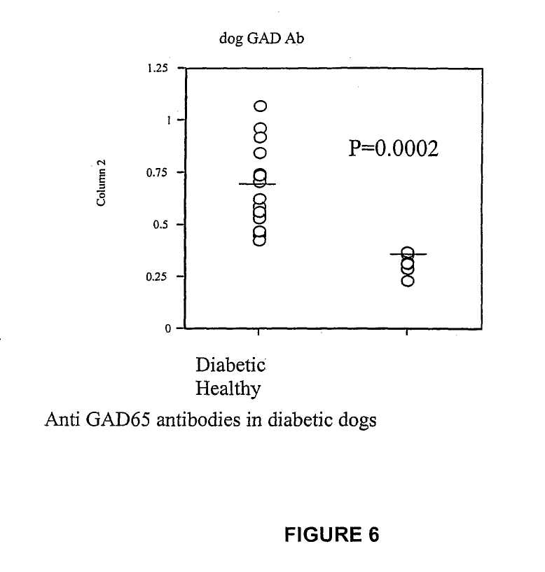

Dogs with recent onset of insulin dependent autoimmune diabetes were tested for the presence of serum anti-GAD65 (total) antibodies using ELISA. As demonstrated, the presence of anti-GAD antibodies was highly related to diabetes as no antibodies were detected in non-diabetic normal dogs (FIG. 6). These data suggest the relevance of GAD65 in canine diabetes, and that early detection of anti-GAD65 antibodies in non diabetic dogs might be used to predict diabetic risk as well as monitoring skewing of TH2 subsets.

### Expression of Canine GAD65 in Transgenic Tobacco Plants.

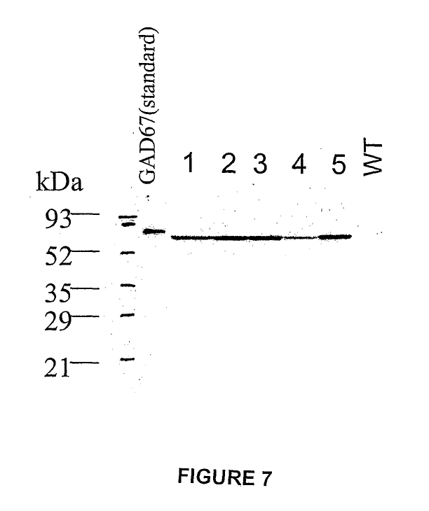

A plant expression vector, containing the entire coding sequence of canine GAD65 under the control of the Cauliflower Mosaic Virus 35S promoter and the polyadenylation signal from the nopaline synthase gene, was constructed and transferred into tobacco plants by the method of Agrobacterium-mediated transformation. Following transformation and selection, transgenic tobacco plants were produced. Integration of canine GAD65 DNA into the nuclear genome of tobacco was confirmed by PCR (polymerase chain reaction) using canine GAD65 specific primers (not shown). Expression of the transferred canine GAD65 at the protein level was determined by Western blot analysis. As shown in FIG. 7, anti-GAD antibody detected a unique band of the expected molecular weight (65 kDa) on Western blots of total leaf extracts prepared from tobacco plants transformed with canine GAD65. In contrast, the same band could not be detected by Western blot analysis in leaf extracts from vector-minus canine GAD transformed tobacco plants. Mouse GAD67 is shown as a size control and is detected by the anti GAD antibody.

### Biological Activity of Plant Cell Culture-Derived Recombinant Canine IL-4.

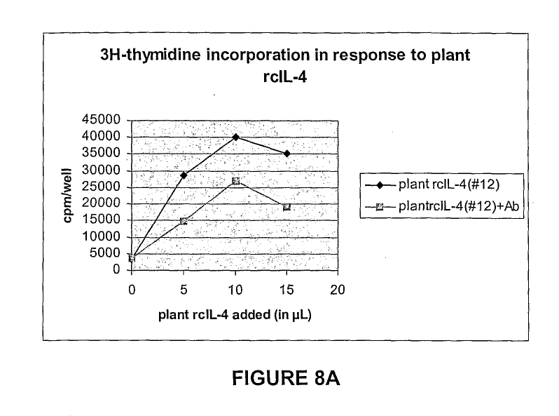

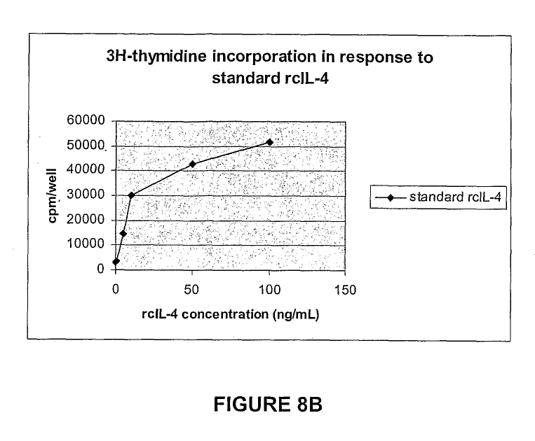

The biological activity of plant-derived rcIL-4 was determined by in vitro bioassay using the cIL-4-dependent cell line (FIGS. 8A, 8B), TF-1. TF-1 cells are a factor-dependent human erythroleukemic cell line that will proliferate in response to canine IL-4. To perform the assay, 6× histidine-tagged rcIL-4 was purified from transgenic tobacco leaf tissue by chromatography on Ni-NTA agarose and used to induce the proliferation of TF-1 cells in comparison with commercial rcIL-4 standard. As shown here, plant-derived rcIL-4 induced TF-1 cells to proliferate in a dose-dependent fashion in culture medium (RPMI 1640), and was comparable to that of rcIL-4 standard. Moreover, co-incubation of plant rcIL-4 with anti-cIL-4 mAb reduced its ability to stimulate the proliferation of TF-1 cells. Taken together, these results suggest that plant-derived rcIL-4 retains its biological and functional authenticity.

### Example C

### Expression of Canine GAD65 and Canine IL-4 in Suspension Cultures

### Dicot Binary Constructs for Expression in Tobacco Cells

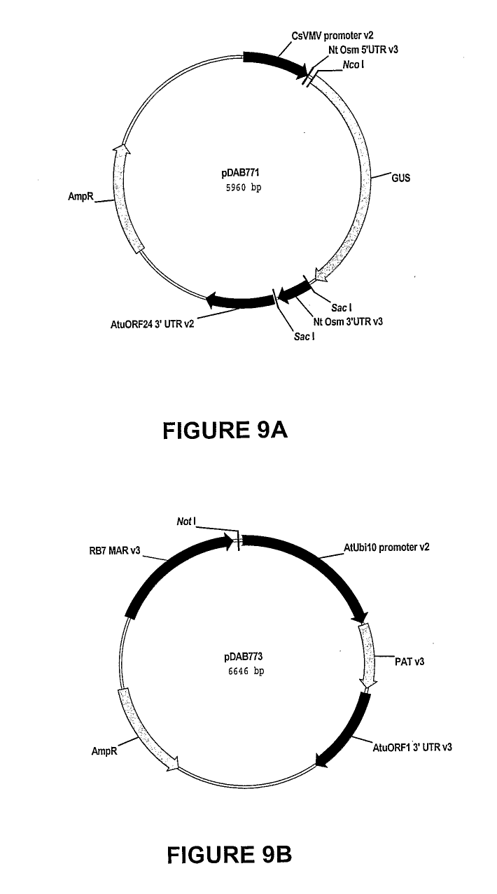

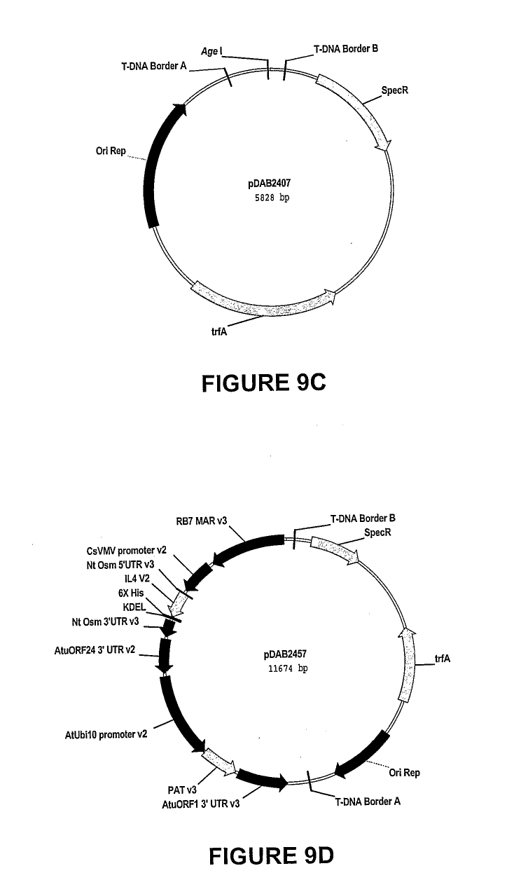

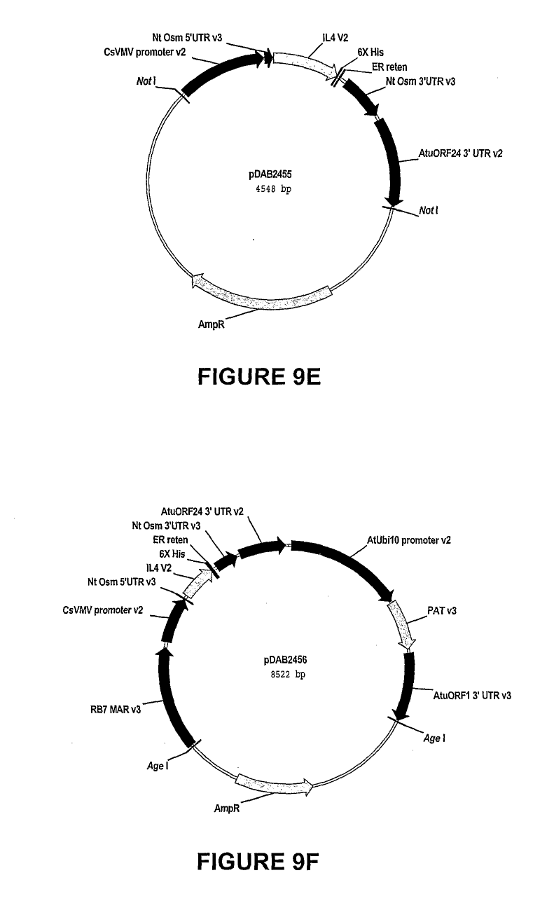

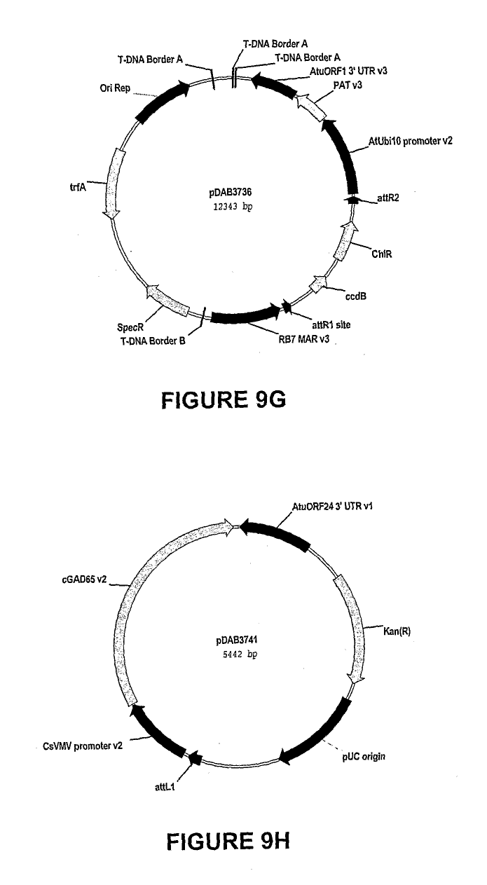

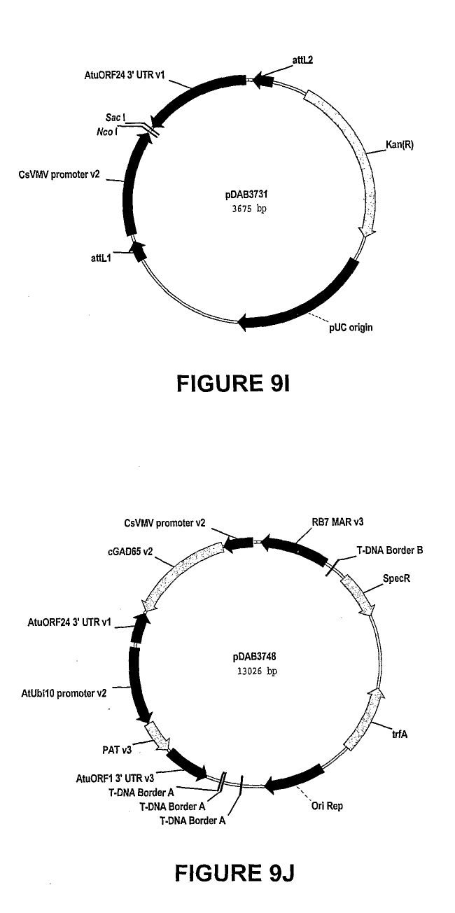

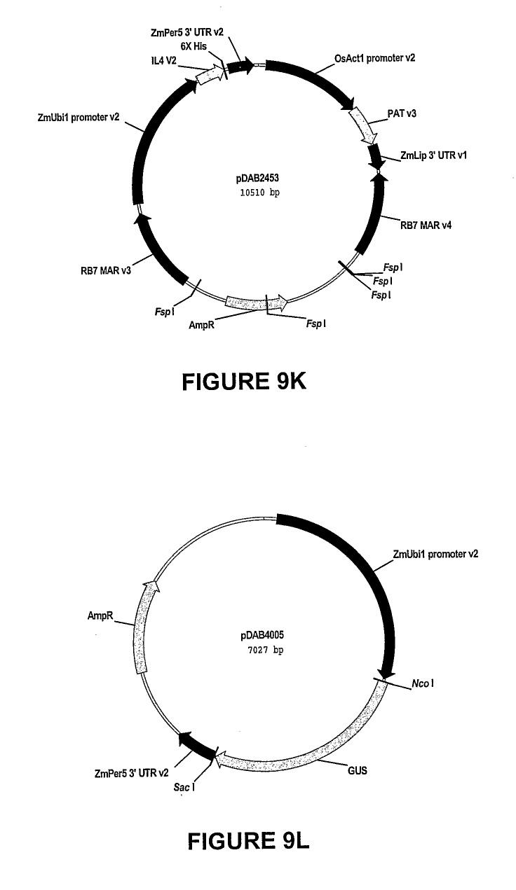

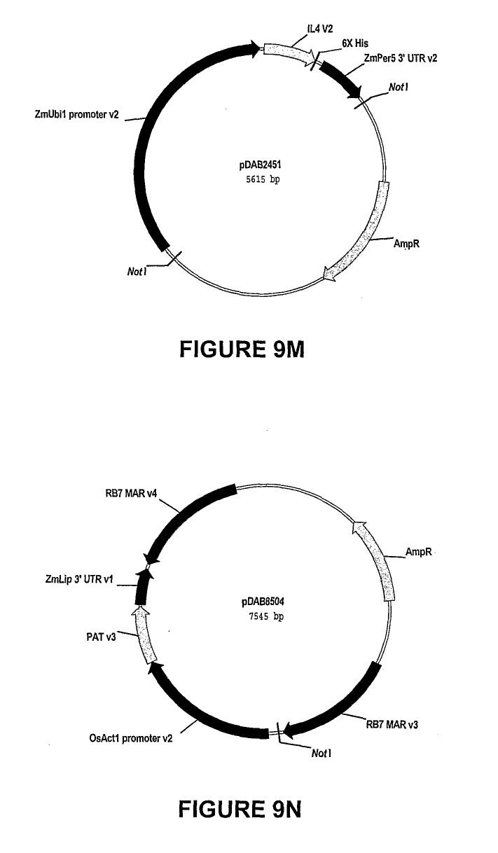

A dicot binary vector, pDAB2457 (Sequence ID No. 1) for Agrobacterium-mediated plant transformation was constructed based on plasmids pDAB771, pDAB773 and pDAB2407. pDAB771 (FIG. 9A) contains the cassava vein mosaic virus promoter described in WO 97/48819 (CsVMV) fused to the 5′ UTR from Nicotiana tabacum osmotin gene (Plant Mol. Bio. 19:577-588 (1992); patent application US 2005102713) and a chimeric 3′ untranslated region consisting of 3′ UTRs from the Nicotiana tabacum osmotin gene (Plant Mol. Bio. 19:577-588 (1992); patent application US 2005102713) and from Agrobacterium tumefaciens plasmid Ti 15955 ORF24 (GenBank accession X00493). Located between the CsVMV promoter and ORF24 3′UTR are unique sites, NcoI and SacI, which were used for inserting genes of interest. pDAB773 (FIG. 9B) contains the RB7 matrix attachment region (MAR) (U.S. Pat. No. 5,773,689; U.S. Pat. No. 5,773,695; U.S. Pat. No. 6,239,328, WO 94/07902, and WO 97/27207) and a transcription unit in which the plant selection marker phosphinothricin acetyl transferase (PAT) (U.S. Pat. Nos. 5,879,903; 5,637,489; 5,276,268; and 5,273,894) is driven by the AtUbi10 promoter (Plant J. 1997. 11(5):1017; Plant Mol. Biol. 1993. 21(5):895; Genetics. 1995. 139(2):921) and flanked, downstream by AtuORF1 3′ UTR (U.S. Pat. No. 5,428,147; Plant Molecular Biology. 1983. 2:335; GenBank accession X00493). A unique NotI site, located between the RB7 MAR gene and the plant AtUbi10 promoter, was used for cloning gene fragments containing the CsVMV promoter, gene of interest, and ORF24 3′UTR. A basic binary vector, pDAB2407 (FIG. 9C) allows for Age1/AgeI ligation of the genes of interest and selectable marker expression cassettes between the T-DNA borders of the Agrobacterium binary vector.

The IL-4 dicot binary vector, pDAB2457 (FIG. 9D), encodes a canine interleukin-4 protein with endoplasmic reticulum (ER) targeting (native) and ER retention signals (Sequence ID NO. 2, Table B). More specifically, the plant transcription unit includes: T-DNA Border B/RB7 MAR v3/CsVMV promoter v2-Nt Osm 5′ UTR v3/IL-4 v2-KDEL/Nt Osm 3′ UTR v3-Atu ORF24 3′ UTR v2::AtUbi10 promoter v2/PAT v3/AtuORF1 3′ UTR v3::T-DNA Border A. The chemically synthesized IL-4 gene contained in DASPICO13 was obtained from PICOSCRIPT in Stratagene's Bluescript vector. A modified version of the gene was produced using PCR and included the IL-4 gene flanked by a 6 histidine tag and an ER retention signal (KDEL). The NcoI/SacI fragment was then inserted into pDAB771 plasmid at the NcoI and SacI sites, resulting in intermediate vector pDAB2455 (FIG. 9E). The CsVMV promoter expression cassette containing IL4 v2-KDEL/ORF24 3′UTR was removed from pDAB2455 with NotI and was ligated in the NotI site of pDAB773, downstream of the RB7 MARv3 gene and upstream of the AtUbi10 promoter v2/PAT v3/AtuORF1 3′UTR selectable marker cassette forming the plant transcription unit (PTU) in intermediate vector pDAB2456 (FIG. 9F). The PTU components were then excised from pDAB2456 using AgeI digestion and ligated in reverse orientation at the AgeI site of pDAB2407 which resulted in the final dicot binary vector, pDAB2457, where the PTU cassette is flanked by T-DNA borders A and B.

### Gateway™ Dicot Binary Construct

Invitrogen's Gateway™ Technology was used for constructing vectors for expression of cGAD65 in tobacco cells. Both the destination and donor vectors were made following Invitrogen's Gateway™ Technology protocol. One destination vector, pDAB3736 (FIG. 9G), and a donor vector, pDAB3741 (FIG. 8), were used to create the cGAD65 binary construct.

Destination vector, pDAB3736, was derived from pDAB2407 and contains attR sites, which recombine with an entry clone in an LR clonase reaction to generate an expression clone (Invitrogen Gateway Technology). It also contains multiple copies of Border A and Border B of the binary vector. Within the border regions, there are an RB7 matrix attachment region (MAR) and Gateway™ cloning sites attR1 and attR2. Entry vector, pDAB3731 (FIG. 9H) contains the attL sites which are used to clone gene fragments that do not contain att sites to generate entry clones (Invitrogen Gateway Technology). pDAB3931 contains the CsVMV v2 promoter and ORF24 3′UTR v1 cassette. Located between the promoter and UTR are NcoI and SacI sites, where the gene of interest is inserted. The cassette is flanked by Gateway™ cloning sites attL1 and attL2.

Gateway™ GAD655 binary vector, pDAB3748 (FIG. 9; Sequence ID NO.3. Table C), contains the PTU cassette: T-DNA Border B:: RB7 MAR v3::CsVMV promoter v2/cGAD v2/Atu ORF24 3′ UTR v2::AtUbi10 promoter v2/PAT v3/Atu ORF1 3′ UTR v3:: Multiple T-DNA Border A. The chemically synthesized cGAD65 gene (native cGAD65-Sequence ID NO. 4; modified cGAD65-Sequence ID NO. 5), which was optimized for plant expression, was excised from pDASPICO27 using NcoI and SacI. The cGAD65 fragment was ligated into the NcoI/SacI sites of pDAB3931 to form the entry clone, pDAB3741. pDAB3741 was transferred into pDAB3736 using LR Clonase to form pDAB3748.

### Monocot Constructs for Expression in Rice Cells

Rice transformation was done using purified DNA fragments. The expression cassette was flanked by FspI sites to allow for purification of the expression cassette from the vector backbone. The expression cassette in pDAB2453 (FIG. 9K; Sequence ID NO.6, Table F) was comprised of a promoter from the maize ubiquitin gene (ZmUbi1 v2; Plant Mol. Biol. 1994. 26(3). 1007; U.S. Pat. No. 5,614,399), modified to remove an NcoI site, and the 3′ UTR region from a maize peroxidase gene (ZmPer5 3′ UTR v2; U.S. Pat. No. 6,699,984). The selectable marker gene cassette included in pDAB2453 was the PAT gene (described above) flanked by the rice actin gene promoter (OsAct1 v2; Mol. Gen. Genet. 1991. 231:150.; GenBank accessions S44221 and X63830;), modified to remove a SacI site and the 3′ UTR from a maize lipase gene (ZmLip 3′ UTR v2; GenBank accession L35913.). The chemically synthesized IL-4 gene was contained in DASPICO13 and was obtained from PICOSCRIPT in Stratagene's Bluescript vector. A modified version of the gene was produced using PCR to create the IL-4 gene flanked by a 6 histidine tag (Sequence ID N0.7, Table 6). The NcoI/SacI fragment from the PCR product was then inserted into pDAB4005 (FIG. 9L) at the NcoI and SacI sites, resulting in intermediate vector pDAB2451 (FIG. 9M). The ZmUbi1 promoter expression cassette containing IL4 v2-KDEL/ZmPer5 3′UTR was removed from pDAB2451 with NotI and was ligated into the NotI site of pDAB8504 (FIG. 9N), upstream of the OsAct1 promoter v2/PAT v3/ZmLip 3′ UTR v2 selectable marker cassette forming the plant transcription unit (PTU) in pDAB2453. The PTU components were then excised from pDAB2453 using FseI digestion and purified for rice transformation experiments.

Production of T-309 Rice Suspensions Stably Transformed with DDAB2453 containing the cIL-4 Gene

Starting material for rice transformations was T309 rice suspension cells maintained in liquid AA media (AA Custom Mix PhytoTechnology Laboratories L.L.C. catalog number CM024), by transferring 8 ml of settled cell volume and 28 ml of conditioned media (media recovered from suspension cultures) into 80 ml of fresh AA cell culture media in 500 ml flasks every three and a half days. Flasks were maintained on a rotary shaker at 28° C. and 125 rpm. WHISKERS™ experiments were initiated by transferring 9 ml of settled cell volume and 27 ml of conditioned media into 80 ml of fresh AA liquid media. Two 500-ml flasks were maintained on a rotary shaker at 125 rpm and 28° C. for 24 hours prior to treatment.

On the day of treatment, the cells were given an osmotic pre-treatment of 30 minutes by drawing off the conditioned media and replacing it with 72 ml of AA liquid media containing 0.25 M sorbitol and 0.25 M mannitol. Following osmotic treatment, the two flasks were pooled into a sterile 250 ml IEC centrifuge bottle (Fisher Scientific catalog number 05-433B). Once the cells had settled, the osmotic media was removed leaving approximately 50 ml of settled cells and media at the bottom of the bottle. Osmotic media was saved to be used during recovery described below.

Whiskering was carried out by adding 8100 μl of freshly prepared 5% Whiskers Suspension (Silar SC-9, Advanced Composite Materials Corp, Greer, S.C.) and 170 μg of plasmid DNA, pDAS2453. The bottle was placed in the modified paint mixer (Red Devil Equipment Co., Minneapolis, Minn.) and agitated on high for 10 seconds after which cells were returned to a 1 L flask with conditioned media and 208 ml of fresh AA liquid media was added. Cells were allowed to recover for 2 hours on a rotary shaker at 125 rpm and 28° C.

Following recovery, 1 ml aliquots of cell suspension were evenly dispensed on sterile, 55 mm number 4 filter paper discs (Whatman International Ltd.) resting on a 60×20 mm Petri dishes containing semi-solid AA media (AA Custom Mix PhytoTechnology Laboratories L.L.C. catalog number CM024 plus 2.5 g/L Gelrite, Sigma-Aldrich catalog number G 9110) and incubated at 28° C. in the dark for three days. After three days, filters with cells were transferred to fresh semi-solid D2[−]P media (N6 Salts catalog number C1416 PhytoTechnology Laboratories, MS/N6 vitamins, 1 g/Ltryptophan, 30 g/L sucrose, 5 mg/L 2,4-D, 2.5 g/L Gelrite, Sigma-Aldrich catalog number G1910, and 3.0 mg/L Herbiace Meiji, Toyoko, Japan) and incubated in the dark at 28° C. for 2 weeks. Filters were transferred to fresh D2[−]P media every 2 weeks until isolates appeared. Calli were placed on semisolid AA media containing 5 mg/L Herbiace and sub-cultured every 2 weeks. Expression analysis was preformed on selected events.

Production of Transgenic Nicotiana tabacum Events Transformed with pDAB2457 containing the cIL-4 Gene

Four days prior to transformation, a 1 week old NT-1 culture was sub-cultured to fresh medium by adding 2 ml of the NT-1 culture or 1 ml of packed cells into 40 ml NT-1 B media. The sub-cultured suspension was maintained in the dark at 25±1° C. on a shaker at 125 rpm.

NT-1 B Medium

| Reagent | Per liter | |
|---|---|---|
| MS salts (10X) | 100 | ml |
| MES | 0.5 | g |
| Thiamine-HCl (1 mg/ml) | 1 | ml |
| Myo-inositol | 100 | mg |
| K₂HPO₄ | 137.4 | mg |
| 2,4-D (10 mg/ml) | 222 | μl |
| Sucrose | 30 | g |
| pH to 5.7 ± 0.03 | | |

Thiamine-HCl (1 mg/ml)(1 liter)
Thiamine HCl (Vit B1) - 0.1 g
2,4-D (10 mg/ml)
Stock solution purchased from Phytotechnology Laboratories

A 50% glycerol stock of Agrobacterium tumefaciens containing the expression vector of interest was used to initiate a liquid culture directly by adding 20-500 μl of the bacteria to 30 ml YEP liquid containing 50 mg/L spectinomycin. The bacterial culture was incubated in the dark at 28° C. in an incubator shaker at 150-200 rpm.

YEP Medium

| Reagent | Per liter |
|---|---|
| Yeast extract | 10 g |
| Peptone | 10 g |
| NaCl | 5 g |
| Sucrose | 10 g |

Four milliliters of the tobacco suspension was transferred into each of 10, 100×25 mm Petri plates. For the treated plates, 100 μl of Agrobacterium suspension at OD₆₀₀=1.5±0.2 was added to each of the 9 plates, keeping one plate as an untreated control. The plates were swirled to mix, wrapped in parafilm and cultured in the dark at 25±1° C. without shaking.

Following the co-cultivation, all liquid was removed and the cells were resuspended in 8 ml NTC medium (NT-1 medium containing 500 mg/L carbenicillin, added after autoclaving). One milliliter aliquots of suspension were distributed to each of 8 Petri plates (100×25 mm) containing NTC+B10 medium (NTC medium solidified with 8 g/l TC Agar supplemented with 10 mg/l bialaphos, added after autoclaving). All selection plates, either wrapped with parafilm or left unwrapped, were maintained in the dark at 28° C. Before wrapping, liquid was removed from any plates that were excessively wet. After 2 to 6 weeks, putative transformants appeared as small clusters of callus on a background of dead, non-transformed cells. They were selected and transferred to fresh NTC+B10. The plates were left unwrapped and cultured in the dark at 28±1° C. Portions of each putative transformant was collected for analysis.

### Extraction of Callus Samples

For western analysis, callus samples are extracted directly in SDS-PAGE gel loading buffer. Two hundred microliters of 2× Laemmli sample buffer (with DTT as the reducing agent) was added to 200 μl of callus tissue. Two steel BBs (Daisy 4.5 mm) were added to each tube and the tubes were shaken for 2 minutes in a Klecko tissue disrupter. After heating for 5-10 minutes at 95° C., the tubes were centrifuged in a microfuge for 10 minutes. The samples were loaded on gels for western analysis.

### Western Analyses of Transgenic Callus Events

Samples for SDS-PAGE were prepared as above for whole cell extracts or by adding loading buffer (4× Laemili Sample Buffer with DTT) and heating for 5 minutes (90-100° C.). Gels (Invitrogen NuPAGE 4-12% Bis-Tris Gel) were run using MES Running Buffer (Invitrogen catalog number NP0002-02). Molecular weight standards (SeeBlue Plus2, MagicMark XP SeeBlue Plus2; catalog numbers LC5925 and LC5602, respectively) and appropriate volume of samples were loaded. The gels were run at 200V for 30-45 minutes. The membranes (0.2 μm nitrocellulose membrane; Invitrogen catalog number LC2000) and pads were soaked for 10-30 min in 10% Methanol Transfer Buffer (NuPAGE Transfer Buffer catalog number NP0006).

The blot module was assembled according to manufacturer directions and blots were transferred at 30V for approximately 1 hour. After transfer, the membranes were rinsed with water and blocked for at least 30 minutes at room temperature with agitation in block solution (WesternBreeze Blocker/Diluent Invitrogen catalog number WB7050). The blots were incubated at least 1 hr in primary antibody in block solution. The membrane was washed 3 times for 5 min each in wash solution (WesternBreeze Wash Solution catalog number WB7003). Treatment with the secondary antibody was similar except the incubation was for at least 30 min. The membrane was washed 3 times for 5 min each in wash solution followed by 2 washes for 2 min each in water prior to adding substrate.

For IL-4 western blots, the standard was recombinant canine IL-4 (R&D Systems catalog number 754-CL); the primary antibody (diluted to 1 ug/ml) was anti-canine IL-4 antibody (R&D Systems catalog number AF 754); the secondary antibody was rabbit anti-goat IgG HRP conjugated (Sigma catalog number A5420) diluted 1:5000. Western immunodetection was done using WesternBreeze Kit (Invitrogen catalog number WB7050) and the Pierce SuperSignal West Pico Luminol Enhancer and Stable Peroxide Solution mixed in equal parts (Pierce catalog number 34080) for detection. The blots were exposed to X-ray film to determine IL-4 expression in the transgenic calli.

For GAD65 western blots, the standard was rhGAD65 (Diamyd Diagnostics catalog number 10-65702-01); the primary antibody was anti-GAD65 (Sigma catalog number G1166) diluted 1:2000; the secondary antibody was goat anti-mouse IgG AP (KPL catalog number 075-1806) diluted 1:1000. The western immunodetection was done using WesternBreeze Kit (Invitrogen catalog number WB7050) and the NBT/BCIP Phosphatase Substrate (KPL catalog number 50-81-08) for detection. The blots were exposed to substrate for 5-10 minutes to determine GAD65 expression in the transgenic calli.

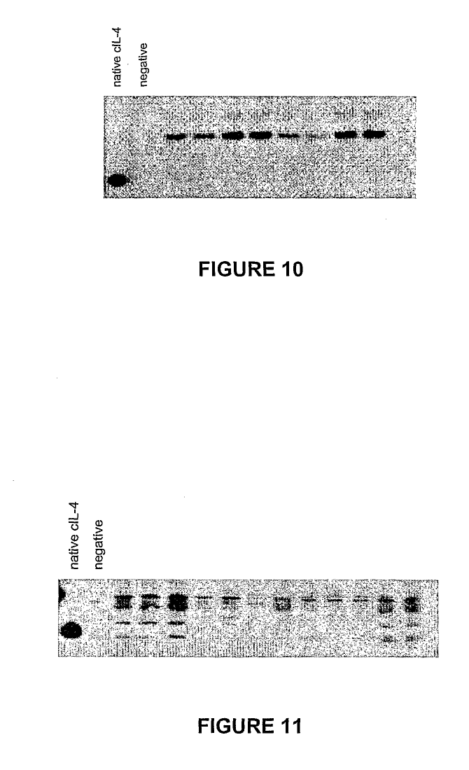

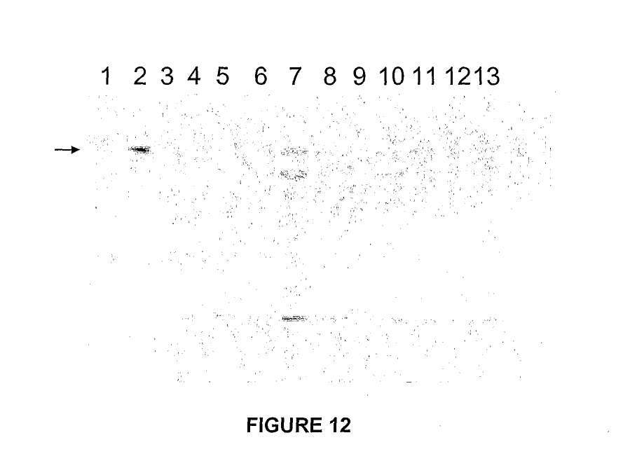

Western analysis demonstrated that canine cIL-4 is expressed in both rice and tobacco cells (FIGS. 10 and 11). As evident from its higher molecular weight relative to the cIL-4 reference protein, the transgenic cIL-4 appears to be post-translationally modified. Canine GAD65 targeted to the cytoplasm is expressed in tobacco cells (FIG. 12). The molecular weight of the transgenic protein is similar to or higher than the cGAD65 reference. Degradation products are also apparent in the western blot.

Characterization of cIL-4

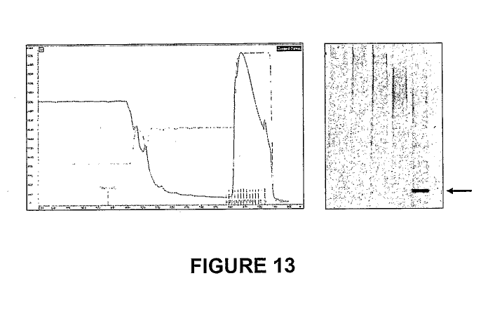

Transgenic cIL-4 was further characterized by extraction of the protein from transgenic tobacco calli. Tissue was ground in liquid nitrogen and ˜10 volumes per weight (ml/g tissue) of 2×PBST (Sigma P3563), 1 M urea, 10% glycerol, 2 mM imidazole, 1 mM PMSF, and 1% protease cocktail inhibitor (Sigma P9599) was added. The suspension was stirred at 4° C. for 30 min. After clarification by centrifugation followed by filtration, the solution was loaded on a Hi-Trap Nickel column (GE Healthcare 17-5247-01) and allowed to recirculate for ˜2 hrs at 2.5 mL/min. The column was washed with 2×PBST, 40 mM imidazole, pH 8.4 and the bound protein was eluted with 20 mM NaHPO₄, 500 mM NaCl and 500 mM imidazole, pH 7.4. The fractions containing cIL-4, as determined by western blot analysis, were combined and loaded on a 100 ml Superose 6 16/50 sizing column (GE Healthcare 17-0489-01) column. Protein was eluted in PBS, pH 7.4 and tested in the in vitro IL-4 activity assay. Samples of the fractions were separated by SDS-PAGE and the major protein band eluted was analyzed by MALDI-TOF to confirm its identity as IL-4 (data not shown). cIL-4 produced in transgenic tobacco callus was purified as described above. The chromatograph of the Hi-Trap Nickel column is shown in FIG. 13, with the fractions retained for further purification. SDS-PAGE analysis of the fractions eluted from the Superose 6 column identified a major protein band (arrow) that corresponded to cIL-4 as determined by western blot and MALDI-TOF analysis.

TABLE A
Sequence ID 1: pDAB2457

ccggttaggatccggtgagtaatattgtacggctaagagcgaatttggcctgtagacctcaattgcgagctttctaatttc
aaactattcgggcctaacttttggtgtgatgatgctgactggcaggatatataccgttgtaatttgagctcgtgtgaataa
gtcgctgtgtatgtttgtttgattgtttctgttggagtgcagcccatttcaccggacaagtcggctagattgatttagccctg
atgaactgccgaggggaagccatcttgagcgcggaatgggaatggatcgaaccgggagcacaggatgacgcctaac
aattcattcaagccgacaccgcttcgcggcgcggcttaattcaggagttaaacatcatgagggaagcggtgatcgccga
agtatcgactcaactatcagaggtagttggcgtcatcgagcgccatctcgaaccgacgttgctggccgtacatttgtacg
gctccgcagtggatggcggcctgaagccacacagtgatattgatttgctggttacggtgaccgtaaggcttgatgaaac
aacgcggcgagctttgatcaacgaccttttggaaacttcggcttcccctggagagagcgagattctccgcgctgtagaag
tcaccattgttgtgcacgacgacatcattccgtggcgttatccagctaagcgcgaactgcaatttggagaatggcagcgc
aatgacattcttgcaggtatcttcgagccagccacgatcgacattgatctggctatcttgctgacaaaagcaagagaaca
tagcgttgccttggtaggtccagcggcggaggaactctttgatccggttcctgaacaggatctatttgaggcgctaaatg
aaaccttaacgctatggaactcgccgcccgactgggctggcgatgagcgaaatgtagtgcttacgttgtcccgcatttgg
tacagcgcagtaaccggcaaaatcgcgccgaaggatgtcgctgccgactgggcaatggagcgcctgccggcccagta
tcagcccgtcatacttgaagctaggcaggcttatcttggacaagaagatcgcttggcctcgcgcgcagatcagttggaa
gaatttgttcactacgtgaaaggcgagatcaccaaggtagtcggcaaataatgtctaacaattcgttcaagccgacgcc
gcttcgcggcgcggcttaactcaagcgttagagagctggggaagactatgcgcgatctgttgaaggtggttctaagcct
cgtacttgcgatggcatttcgatcgaaaggggtacaaattcccactaagcgctcgggggctgagaaagcccagtaagg
aaacaactgtaggttcgagtcgcgagatcccccggaaccaaaggaagtaggttaaacccgctccgatcaggccgagc
cacgccaggccgagaacattggttcctgtaggcatcgggattggcggatcaaacactaaagctactggaacgagcaga
agtcctccggccgccagttgccaggcggtaaaggtgagcagaggcacgggaggttgccacttgcgggtcagcacggtt
ccgaacgccatggaaaccgcccccgccaggcccgctgcgacgccgacaggatctagcgctgcgtttggtgtcaacacc
aacagcgccacgcccgcagttccgcaaatagcccccaggaccgccatcaatcgtatcgggctacctagcagagcggca
gagatgaacacgaccatcagcggctgcacagcgcctaccgtcgccgcgaccccgcccggcaggcggtagaccgaaat
aaacaacaagctccagaatagcgaaatattaagtgcgccgaggatgaagatgcgcatccaccagattcccgttggaat
ctgtcggacgatcatcacgagcaataaacccgccggcaacgcccgcagcagcataccggcgacccctcggcctcgctg
ttcgggctccacgaaaacgccggacagatgcgccttgtgagcgtccttggggccgtcctcctgtttgaagaccgacagc
ccaatgatctcgccgtcgatgtaggcgccgaatgccacggcatctcgcaaccgttcagcgaacgcctccatgggctttttc
tcctcgtgctcgtaaacggacccgaacatctctggagctttcttcagggccgacaatcggatctcgcggaaatcctgcac
gtcggccgctccaagccgtcgaatctgagccttaatcacaattgtcaattttaatcctctgtttatcggcagttcgtagagc
gcgccgtgcgcccgagcgatactgagcgaagcaagtgcgtcgagcagtgcccgcttgttcctgaaatgccagtaaagc
gctggctgctgaacccccagccggaactgaccccacaaggccctagcgtttgcaatgcaccaggtcatcattgacccag
gcgtgttccaccaggccgctgcctcgcaactcttcgcaggcttcgccgacctgctcgcgccacttcttcacgcgggtggaa
tccgatccgcacatgaggcggaaggtttccagcttgagcgggtacggctcccggtgcgagctgaaatagtcgaacatcc
gtcgggccgtcggcgacagcttgcggtacttctcccatatgaatttcgtgtagtggtcgccagcaaacagcacgacgatt
tcctcgtcgatcaggacctggcaacgggacgttttcttgccacggtccaggacgcggaagcggtgcagcagcgacacc
gattccaggtgcccaacgcggtcggacgtgaagcccatcgccgtcgcctgtaggcgcgacaggcattcctcggccttcg
tgtaataccggccattgatcgaccagcccaggtcctggcaaagctcgtagaacgtgaaggtgatcggctcgccgatag
gggtgcgcttcgcgtactccaacacctgctgccacaccagttcgtcatcgtcggcccgcagctcgacgccggtgtaggtg
atcttcacgtccttgttgacgtggaaaatgaccttgttttgcagcgcctcgcgcgggattttcttgttgcgcgtggtgaaca
gggcagagcgggccgtgtcgtttggcatcgctcgcatcgtgtccggccacggcgcaatatcgaacaaggaaagctgca
tttccttgatctgctgcttcgtgtgtttcagcaacgcggcctgcttggcctcgctgacctgttttgccaggtcctcgccggcg
gtttttcgcttcttggtcgtcatagttcctcgcgtgtcgatggtcatcgacttcgccaaacctgccgcctcctgttcgagacg
acgcgaacgctccacggcggccgatggcgcgggcagggcagggggagccagttgcacgctgtcgcgctcgatcttgg
ccgtagcttgctggaccatcgagccgacggactggaaggtttcgcggggcgcacgcatgacggtgcggcttgcgatgg
tttcggcatcctcggcggaaaaccccgcgtcgatcagttcttgcctgtatgccttccggtcaaacgtccgattcattcaccct
ccttgcgggattgccccgactcacgccggggcaatgtgcccttattcctgatttgacccgcctggtgccttggtgtccagat
aatccaccttatcggcaatgaagtcggtcccgtagaccgtctggccgtccttctcgtacttggtattccgaatcttgccctgc
acgaataccagctccgcgaagtcgctcttcttgatggagcgcatggggacgtgcttggcaatcacgcgcaccccccggc
cgttttagcggctaaaaaagtcatggctctgccctcgggcggaccacgcccatcatgaccttgccaagctcgtcctgcttc
tcttcgatcttcgccagcagggcgaggatcgtggcatcaccgaaccgcgccgtgcgcgggtcgtcggtgagccagagtt
tcagcaggccgcccaggcggcccaggtcgccattgatgcgggccagctcgcggacgtgctcatagtccacgacgcccg
tgattttgtagccctggccgacggccagcaggtaggccgacaggctcatgccggccgccgccgccttttcctcaatcgct
cttcgttcgtctggaaggcagtacaccttgataggtgggctgcccttcctggttggcttggtttcatcagccatccgcttgcc
ctcatctgttacgccggcggtagccggccagcctcgcagagcaggattcccgttgagcaccgccaggtgcgaataagg
gacagtgaagaaggaacacccgctcgcgggtgggcctacttcacctatcctgcccggctgacgccgttggatacaccaa
ggaaagtctacacgaaccctttggcaaaatcctgtatatcgtgcgaaaaaggatggatataccgaaaaaatcgctataa
tgaccccgaagcagggttatgcagcggaaaagatccgtcgaccctttccgacgctcaccgggctggttgccctcgccgc
tgggctggcggccgtctatggccctgcaaacgcgccagaaacgccgtcgaagccgtgtgcgagacaccgcggccgcc
ggcgttgtggatacctcgcggaaaacttggccctcactgacagatgaggggcggacgttgacacttgaggggccgact
cacccggcgcggcgttgacagatgaggggcaggctcgatttcggccggcgacgtggagctggccagcctcgcaaatc
ggcgaaaacgcctgattttacgcgagtttcccacagatgatgtggacaagcctggggataagtgccctgcggtattgac
acttgaggggcgcgactactgacagatgaggggcgcgatccttgacacttgaggggcagagtgctgacagatgaggg
gcgcacctattgacatttgaggggctgtccacaggcagaaaatccagcatttgcaagggtttccgcccgtttttcggccac
cgctaacctgtcttttaacctgcttttaaaccaatatttataaaccttgtttttaaccagggctgcgccctgtgcgcgtgaccg
cgcacgccgaaggggggtgcccccccttctcgaaccctcccggcccgctaacgcgggcctcccatccccccaggggctg
cgcccctcggccgcgaacggcctcaccccaaaaatggcagccaagcttgcttggtcgttccggtacgtaccgtgaacgt
cggctcgattgtacctgcgttcaaatactttgcgatcgtgttgcgcgcctgcccggtgcgtcggctgatctcacggatcga
ctgcttctctcgcaacgccatccgacggatgatgtttaaaagtcccatgtggatcactccgttgccccgtcgctcaccgtgt
tggggggaaggtgcacatggctcagttctcaatggaaattatctgcctaaccggctcagttctgcgtagaaaccaacatg
caagctccaccgggtgcaaagcggcagcggcggcaggatatattcaattgtaaatggcttcatgtccgggaaatctaca
tggatcagcaatgagtatgatggtcaatatggagaaaaagaaagagtaattaccaattttttttcaattcaaaaatgtag
atgtccgcagcgttattataaaatgaaagtacattttgataaaacgacaaattacgatccgtcgtatttataggcgaaagc
aataaacaaattattctaattcggaaatctttatttcgacgtgtctacattcacgtccaaatgggggccagatcagaattcc
tcgagtttaaacggatcctaaccggtgactagaaacccggccgctatgtattacaccataatatcgcactcagtctttcatc
tacggcaatgtaccagctgatataatcagttattgaaatatttctgaatttaaacttgcatcaataaatttatgtttttgcttg
gactataatacctgacttgttattttatcaataaatatttaaactatatttctttcaagatatcattctttacaagtatacgtgtt
taaattgaataccataaatttttatttttcaaatacatgtaaaattatgaaatgggagtggtggcgaccgcaagcacactt
caattcctataacggaccaaatcgcaaaaattataataacatattatttcatcctggattaaaagaaagtcaccggggatt
attttgtgacgccgattacatacggcgacaataaagacattggaaatcgtagtacatattggaatacactgattatattag
tgatgaatacatactttaatatccttacgtaggatcaacatatcttgttacaatcggacacttttgcttcatcccgctaacac
ctctgcaccttagaccaagcgcttccacaaggaactgagagccatagcccacctcaccttgggttcctttggccgcctgtc
tttctgaaagagagccttgcccaccgcaactatttcaacacagataggatcaacccgggatggcgctaagaagctattg
ccgccgatctgggcgcctatctagtcaagggcgaattccagcacactggcggccgttactagtggatccgagctctaag
ctcataagctcataagctcaagctcagggtacctcagatctgggtaactggcctaactggccttggaggagctggcaact
caaaatccctttgccaaaaaccaacatcatgccatccaccatgcttgtatccagctgcgcgcaatgtaccccgggctgtgt
atcccaaagcctcatgcaacctaacagatggatcgtttggaaggcctataacagcaaccacagacttaaaaccttgcgc
ctccatagacttaagcaaatgtgtgtacaatgtggatcctaggcccaacctttgatgcctatgtgacacgtaaacagtact
ctcaactgtccaatcgtaagcgttcctagccttccagggcccagcgtaagcaataccagccacaacaccctcaacctcag
caaccaaccaagggtatctatcttgcaacctctctagatcatcaatccactcttgtggtgtttgtggctctgtcctaaagttc
actgtagacgtctcaatgtaatggttaacgatatcacaaaccgcggccatatcagctgctgtagctggcctaatctcaact
ggtctcctctccggagaagccatggttggatccttacctgttaatcagaaaaactcagattaatcgacaaattcgatcgca
caaactagaaactaacaccagatctagatagaaatcacaaatcgaagagtaattattcgacaaaactcaaattatttga
acaaatcggatgatatttatgaaaccctaatcgagaattaagatgatatctaacgatcaaacccagaaaatcgtcttcga
tctaagattaacagaatctaaaccaaagaacatatacgaaattgggatcgaacgaaaacaaaatcgaagattttgaga
gaataaggaacacagaaatttaccttgatcacggtagagagaattgagagaaagtttttaagattttgagaaattgaaa
tctgaattgtgaagaagaagctttgggtattgttttatagaagaagaagaagaaaagacgaggacgactaggtcacga
gaaagctaaggcggtgaagcaatagctaataataaaatgacacgtgtattgagcgttgtttacacgcaaagttgtttttg
gctaattgccttatttttaggttgaggaaaagtatttgtgctttgagttgataaacacgactcgtgtgtgccggctgcaacc
actttgacgccgtttattactgactcgtcgacaaccacaatttctaacggtcgtcataagatccagccgttgagatttaacg
atcgttacgatttatatttttttagcattatcgttttattttttaaatatacggtggagctgaaaattggcaataattgaaccgt
gggtcccactgcattgaagcgtatttcgtattttctagaattcttcgtgctttatttcttttcctttttgtttttttttgccatttatct
aatgcaagtgggcttataaaatcagtgaatttcttggaaaagtaacttctttatcgtataacatattgtgaaattatccattt
cttttaattttttagtgttattggatatttttgtatgattattgatttgcataggataatgacttttgtatcaagttggtgaacaa
gtctcgttaaaaaaggcaagtggtttggtgactcgatttattcttgttatttaattcatatatcaatggatcttatttggggcc
tggtccatatttaacactcgtgttcagtccaatgaccaataatattttttcattaataacaatgtaacaagaatgatacaca
aaacattctttgaataagttcgctatgaagaagggaacttatccggtcctagatcatcagttcatacaaacctccatagag
ttcaacatcttaaacaagaatatcctgatccgttgacctgcaggcggggtttaaacatttaaatttaattaagcggccgcg
gccggccgagcataatttttattaatgtactaaattactgttttgttaaatgcaattttgctttctcgggattttaatatcaaaa
tctatttagaaatacacaatattttgttgcaggcttgctggagaatcgatctgctatcataaaaattacaaaaaaattttatt
tgcctcaattattttaggattggtattaaggacgcttaaattatttgtcgggtcactacgcatcattgtgattgagaagatca
gcgatacgaaatattcgtagtactatcgcgataatttatttgaaaattcatatgaaaagcaaacgttacatgaattgatga
aacaatacaaagacagataaagccacgcacatttaggatattggccgagattactgaatattgagtaagatcacggaa
tttctgacaggagcatgtcttcaattcagcccaaatggcagttgaaatactcaaaccgccccatatgcaggagcggatca
ttcattgtttgtttggttgcctttgccaacatgggagtccaaggtttggtgaccgcatgcgagctcaagggcgaatttcgag
cttaagactttactaaaacttcaaaagaaaaacaatataaaaacgataatccaaatgcattattgatctatataacatcaa
gacaaaaatacatatgtgactcttattcaggtcttaggtttattacagcaaagatcatgacttgatcacttcaaacaaagt
acgtaactataaaaacgagtcaaatagattgtcttacactaacgtgtcgatagaataatttgaccaaaaggtgatcttatt
acagaaatagccactgagctctaggtgattaagctaactactcaaagttcatccttctcagaatggtgatggtgatgatgt
ctgtaatacttcttctgcattatcactttcagcctttccagaaagtctttcaatgtagatttcttgatttcattcatgctacaggt
tttgtttgccattgagctgagatttcgataaagacccctcaagtatctattggagcagttgtgggtgtagatttgtctgagg
acagtggcagcacggcagaaaatctctttgtctgaagtgttcttaggagcagtgaaaacatccttcacagtcagctccat
gcaactatcattccttgctgtcaaaatgttcaacattttgattatctccttaatggtgatattgaagttgtgtccatgaacaaa
tgtggaggtgagggcaaggagacagacaagagtcggaatcagttgtgatgtgaggcccatgggttgttggaaatttttt
ttaacaagttgggttgttggataagatctacaaacttacaaatttctctgaagttgtatcctcagtacttcaaagaaaatag
cttacaccaaattttttcttgttttcacaaatgccgaacttggttccttatataggaaaactcaagggcaaaaatgacacgg
aaaaatataaaaggataagtagtgggggataagattcctttgtgataaggttactttccgcccttacattttccaccttaca
tgtgtcctctatgtctctttcacaatcaccgaccttatcttcttcttttcattgttgtcgtcagtgcttacgtcttcaagattctttt
cttcgcctggttcttctttttcaatttctacgtattcttcttcgtattctggcagtataggatcttgtatctgtacattcttcattttt
gaacataggttgcatatgtgccgcatattgatctgcttcttgctgagcttacataatacttccatagtttttcccgtaaacatt
ggattcttgatgctacatcttggataattaccttctgggtttaaacaagctttgcggccgcttgcccgggcatggcgcgcct
taattaagcggtggccactattttcagaagaagttcccaatagtagtccaaaatttttgtaacgaagggagcataatagtt
acatgcaaaggaaaactgccattctttagaggggatgcttgtttaagaacaaaaaatatatcactttcttttgttccaagtc
attgcgtatttttttaaaaatatttgttccttcgtatatttcgagcttcaatcactttatggttcattgtattctggctttgctgta
aatcgtagctaaccttcttcctagcagaaattattaatacttgggatatttttttagaatcaagtaaattacatattaccacc
acatcgagctgcttttaaattcatattacagccatataggcttgattcattttgcaaaatttccaggatattgacaacgttaa
cttaataatatcttgaaatattaaagctattatgattaggggtgcaaatggaccgagttggttcggtttatatcaaaatcaa
accaaaccaactatatcggtttggattggttcggttttgccgggttttcagcattttctggttttttttttgttagatgaatatta
ttttaatcttactttgtcaaatttttgataagtaaatatatgtgttagtaaaaattaattttttttacaaacatatgatctattaa
aatattcttataggagaattttcttaataacacatgatatttatttattttagtcgtttgactaatttttcgttgatgtacactttc
aaagttaaccaaatttagtaattaagtataaaaatcaatatgatacctaaataatgatatgttctatttaattttaaattatc
gaaatttcacttcaaattcgaaaaagatatataagaattttgataggttttgacatatgaatatggaagaacaaagagat
tgacgcattttagtaacacttgataagaaagtgatcgtacaaccaattatttaaagttaataaaaatggagcacttcatat
ttaacgaaatattacatgccagaagagtcgcaaatatttctagatattttttaaagaaaattctataaaaagtcttaaagg
catatatataaaaactatatatttatattttggtttggttcgaatttgttttactcaataccaaactaaattagaccaaatata
attgagatttttaatcgcggcccatgatcaca

TABLE B

Sequence ID 2: canine IL-4 optimized for plant
expression with an ER retention signal and a 6
histidine tag

atgggcctcacatcacaactgattccgactcttgtctgtctccttgccctcacctccacatttgttcatggacacaacttcaat
atcaccattaaggagataatcaaaatgttgaacattttgacagcaaggaatgatagttgcatggagctgactgtgaagg
atgttttcactgctcctaagaacacttcagacaaagagattttctgccgtgctgccactgtcctcagacaaatctacaccca
caactgctccaatagatacttgaggggtctttatcgaaatctcagctcaatggcaaacaaaacctgtagcatgaatgaaa
tcaagaaatctacattgaaagactttctggaaaggctgaaagtgataatgcagaagaagtattacagacatcatcacca
tcaccattctgagaaggatgaactt

TABLE C
Sequence ID 3: pDAB3748

aatagcgaaatattaagtgcgccgaggatgaagatgcgcatccaccagattcccgttggaatctgtcggacgatcatca
cgagcaataaacccgccggcaacgcccgcagcagcataccggcgacccctcggcctcgctgttcgggctccacgaaaa
cgccggacagatgcgccttgtgagcgtccttggggccgtcctcctgtttgaagaccgacagcccaatgatctcgccgtcg
atgtaggcgccgaatgccacggcatctcgcaaccgttcagcgaacgcctccatgggctttttctcctcgtgctcgtaaacg
gacccgaacatctctggagctttcttcagggccgacaatcggatctcgcggaaatcctgcacgtcggccgctccaagccg
tcgaatctgagccttaatcacaattgtcaattttaatcctctgtttatcggcagttcgtagagcgcgccgtgcgcccgagcg
atactgagcgaagcaagtgcgtcgagcagtgcccgcttgttcctgaaatgccagtaaagcgctggctgctgaaccccca
gccggaactgaccccacaaggccctagcgtttgcaatgcaccaggtcatcattgacccaggcgtgttccaccaggccgc
tgcctcgcaactcttcgcaggcttcgccgacctgctcgcgccacttcttcacgcgggtggaatccgatccgcacatgaggc
ggaaggtttccagcttgagcgggtacggctcccggtgcgagctgaaatagtcgaacatccgtcgggccgtcggcgaca
gcttgcggtacttctcccatatgaatttcgtgtagtggtcgccagcaaacagcacgacgatttcctcgtcgatcaggacct
ggcaacgggacgttttcttgccacggtccaggacgcggaagcggtgcagcagcgacaccgattccaggtgcccaacgc
ggtcggacgtgaagcccatcgccgtcgcctgtaggcgcgacaggcattcctcggccttcgtgtaataccggccattgatc
gaccagcccaggtcctggcaaagctcgtagaacgtgaaggtgatcggctcgccgataggggtgcgcttcgcgtactcc
aacacctgctgccacaccagttcgtcatcgtcggcccgcagctcgacgccggtgtaggtgatcttcacgtccttgttgacg
tggaaaatgaccttgttttgcagcgcctcgcgcgggattttcttgttgcgcgtggtgaacagggcagagcgggccgtgtc
gtttggcatcgctcgcatcgtgtccggccacggcgcaatatcgaacaaggaaagctgcatttccttgatctgctgcttcgt
gtgtttcagcaacgcggcctgcttggcctcgctgacctgttttgccaggtcctcgccggcggtttttcgcttcttggtcgtcat
agttcctcgcgtgtcgatggtcatcgacttcgccaaacctgccgcctcctgttcgagacgacgcgaacgctccacggcgg
ccgatggcgcgggcagggcagggggagccagttgcacgctgtcgcgctcgatcttggccgtagcttgctggaccatcg
agccgacggactggaaggtttcgcggggcgcacgcatgacggtgcggcttgcgatggtttcggcatcctcggcggaaa
accccgcgtcgatcagttcttgcctgtatgccttccggtcaaacgtccgattcattcaccctccttgcgggattgccccgact
cacgccggggcaatgtgcccttattcctgatttgacccgcctggtgccttggtgtccagataatccaccttatcggcaatga
agtcggtcccgtagaccgtctggccgtccttctcgtacttggtattccgaatcttgccctgcacgaataccagctccgcgaa
gtcgctcttcttgatggagcgcatggggacgtgcttggcaatcacgcgcaccccccggccgttttagcggctaaaaaagt
catggctctgccctcgggcggaccacgcccatcatgaccttgccaagctcgtcctgcttctcttcgatcttcgccagcagg
gcgaggatcgtggcatcaccgaaccgcgccgtgcgcgggtcgtcggtgagccagagtttcagcaggccgcccaggcg
gcccaggtcgccattgatgcgggccagctcgcggacgtgctcatagtccacgacgcccgtgattttgtagccctggccga
cggccagcaggtaggccgacaggctcatgccggccgccgccgccttttcctcaatcgctcttcgttcgtctggaaggcag
tacaccttgataggtgggctgcccttcctggttggcttggtttcatcagccatccgcttgccctcatctgttacgccggcggt
agccggccagcctcgcagagcaggattcccgttgagcaccgccaggtgcgaataagggacagtgaagaaggaacac
ccgctcgcgggtgggcctacttcacctatcctgcccggctgacgccgttggatacaccaaggaaagtctacacgaaccct
ttggcaaaatcctgtatatcgtgcgaaaaaggatggatataccgaaaaaatcgctataatgaccccgaagcagggttat
gcagcggaaaagatccgtcgaccctttccgacgctcaccgggctggttgccctcgccgctgggctggcggccgtctatg
gccctgcaaacgcgccagaaacgccgtcgaagccgtgtgcgagacaccgcggccgccggcgttgtggatacctcgcg
gaaaacttggccctcactgacagatgaggggcggacgttgacacttgaggggccgactcacccggcgcggcgttgac
agatgaggggcaggctcgatttcggccggcgacgtggagctggccagcctcgcaaatcggcgaaaacgcctgatttta
cgcgagtttcccacagatgatgtggacaagcctggggataagtgccctgcggtattgacacttgaggggcgcgactact
gacagatgaggggcgcgatccttgacacttgaggggcagagtgctgacagatgaggggcgcacctattgacatttga
ggggctgtccacaggcagaaaatccagcatttgcaagggtttccgcccgtttttcggccaccgctaacctgtcttttaacct
gcttttaaaccaatatttataaaccttgtttttaaccagggctgcgccctgtgcgcgtgaccgcgcacgccgaaggggggt
gcccccccttctcgaaccctcccggcccgctaacgcgggcctcccatccccccaggggctgcgcccctcggccgcgaac
ggcctcaccccaaaaatggcagccaagcttgcttggtcgttccggtacgtaccgtgaacgtcggctcgattgtacctgcg
ttcaaatactttgcgatcgtgttgcgcgcctgcccggtgcgtcggctgatctcacggatcgactgcttctctcgcaacgcca
tccgacggatgatgtttaaaagtcccatgtggatcactccgttgccccgtcgctcaccgtgttggggggaaggtgcacat
ggctcagttctcaatggaaattatctgcctaaccggctcagttctgcgtagaaaccaacatgcaagctccaccgggtgca
aagcggcagcggcggcaggatatattcaattgtaaatggcttcatgtccgggaaatctacatggatcagcaatgagtat
gatggtcaatatggagaaaaagaaagagtaattaccaattttttttcaattcaaaaatgtagatgtccgcagcgttattat
aaaatgaaagtacattttgataaaacgacaaattacgatccgtcgtatttataggcgaaagcaataaacaaattattcta
attcggaaatctttatttcgacgtgtctacattcacgtccaaatgggggccagatcagaattcctcgacacgcgtgggccg
gccggcaggatatattcaattgtaaatcgagctgttggctggctggggcaggatatattcaattgtaaatcaaatt9acg
cttagacaacttaataacacattgcggacgtttttaatgtactgaagtcacatccgtttgatacttgtctaaaattggctgat
ttcgagtgcatctatgcataaaaacaatctaatgacaattattaccaagcatcaatgagatgatgtgtgtgtctatgtgcat
gcgctagcctcgagtttaaacggatcctaaccggtgactagaaacccggccgctatgtattacaccataatatcgcac-tc
agtctttcatctacggcaatgtaccagctgatataatcagttattgaaatatttctgaatttaaacttgcatcaataaatttat
gtttttgcttggactataatacctgacttgttattttatcaataaatatttaaactatatttctttcaagatatcattctttacaa
gtatacgtgtttaaattgaataccataaatttttatttttcaaatacatgtaaaattatgaaatgggagtggtggcgaccgc
aagcacacttcaattcctataacggaccaaatcgcaaaaattataataacatattatttcatcctggattaaaagaaagtc
accggggattattttgtgacgccgattacatacggcgacaataaagacattggaaatcgtagtacatattggaatacact
gattatattagtgatgaatacatactttaatatccttacgtaggatcaacatatcttgttacaatcggacacttttgcttcatc
ccgctaacacctctgcaccttagaccaagcgcttccacaaggaactgagagccatagcccacctcaccttgggttcctttg
gccgcctgtctttctgaaagagagccttgcccaccgcaactatttcaacacagataggatcaacccgggatggcgctaa
gaagctattgccgccgatctgggcgcctatctagtcaagggcgaattccagcacactggcggccgttactagtggatcc
gagctctaagctcataagctcataagctcaagctcagggtacctcagatctgggtaactggcctaactggccttggagg
agctggcaactcaaaatccctttgccaaaaaccaacatcatgccatccaccatgcttgtatccagctgcgcgcaatgtacc
ccgggctgtgtatcccaaagcctcatgcaacctaacagatggatcgtttggaaggcctataacagcaaccacagactta
aaaccttgcgcctccatagacttaagcaaatgtgtgtacaatgtggatcctaggcccaacctttgatgcctatgtgacacg
taaacagtactctcaactgtccaatcgtaagcgttcctagccttccagggcccagcgtaagcaataccagccacaacacc
ctcaacctcagcaaccaaccaagggtatctatcttgcaacctctctagatcatcaatccactcttgtggtgtttgtggctctg
tcctaaagttcactgtagacgtctcaatgtaatggttaacgatatcacaaaccgcggccatatcagctgctgtagctggcc
taatctcaactggtctcctctccggagaagccatggttggatccttacctgttaatcagaaaaactcagattaatcgacaa
attcgatcgcacaaactagaaactaacaccagatctagatagaaatcacaaatcgaagagtaattattcgacaaaactc
aaattatttgaacaaatcggatgatatttatgaaaccctaatcgagaattaagatgatatctaacgatcaaacccagaaa
atcgtcttcgatctaagattaacagaatctaaaccaaagaacatatacgaaattgggatcgaacgaaaacaaaatcga
agattttgagagaataaggaacacagaaatttaccttgatcacggtagagagaattgagagaaagtttttaagattttg
agaaattgaaatctgaattgtgaagaagaagctttgggtattgttttatagaagaagaagaagaaaagacgaggacg
actaggtcacgagaaagctaaggcggtgaagcaatagctaataataaaatgacacgtgtattgagcgttgtttacacgc
aaagttgtttttggctaattgccttatttttaggttgaggaaaagtatttgtgctttgagttgataaacacgactcgtgtgtgc
cggctgcaaccactttgacgccgtttattactgactcgtcgacaaccacaatttctaacggtcgtcataagatccagccgtt
gagatttaacgatcgttacgatttatatttttttagcattatcgttttattttttaaatatacggtggagctgaaaattggcaat
aattgaaccgtgggtcccactgcattgaagcgtatttcgtattttctagaattcttcgtgctttatttcttttcctttttgtttttttt
tgccatttatctaatgcaagtgggcttataaaatcagtgaatttcttggaaaagtaacttctttatcgtataacatattgtga
aattatccatttcttttaattttttagtgttattggatatttttgtatgattattgatttgcataggataatgacttttgtatcaagt
tggtgaacaagtctcgttaaaaaaggcaagtggtttggtgactcgatttattcttgttatttaattcatatatcaatggatct
tatttggggcctggtccatatttaacactcgtgttcagtccaatgaccaataatattttttcattaataacaatgtaacaaga
atgatacacaaaacattctttgaataagttcgctatgaagaagggaacttatccggtcctagatcatcagttcatacaaac
ctccatagagttcaacatcttaaacaagaatatcctgatccgttgacctgcaggcggggtttaaacatttaaatttaattaa
gcggccgcaaccactttgtacaagaaagctgggtcggcgcgcccacccttgcggccgcaaccttggactcccatgttgg
caaaggcaaccaaacaaacaatgaatgatccgctcctgcatatggggcggtttgagtatttcaactgccatttgggctga
attgaagacatgctcctgtcagaaattccgtgatcttactcaatattcagtaatctcggccaatatcctaaatgtgcgtggc
tttatctgtctttgtattgtttcatcaattcatgtaacgtttgcttttcttatgaattttcaaataaattatcgatagtactacgaa
tatttcgtatcgctgatcttctcaatcacaatgatgcgtagtgacccgacaaataatttaagcgtccttaataccaatcctaa
aataattgaggcaaataaaatttttttgtaatttttatgatagcagatcgattctccagcaagcctgcaacaaaatattgtg
tatttctaaatagattttgatattaaaatcccgagaaagcaaaattgcatttaacaaaacagtaatttagtacattaataaa
aattatgctggtgaccgagctctaggtgattaagctaactactcaaagatcttgtccaagtctttcaatctcttcaatgaga
aagtcaatgtcttgatgagttgcagcaggattggagatgaccattcgaaagaagttcaccttgtctcccagtggctggta
gctgaccattgtggtcccatactccatcatccttgccttgatcactggtgccacctttgagagtctgttcatcctctcttcgttg
tcttccagaaccctcaatgacggaggaacataccagaagcagacattggtgtgttgtggcttcccatcaaacaccatctc
gtagccttctcgattcttgataatgctgtacaggtactctgcaagctccaaacacttgtctatgtgagcctcaaacccagtg
gtccctttggctctccacatgagccagagcttgaagacatcaacatgtcgtccacactgcaaggctttgtctccagtgtcat
aggacagatcatagtgtttgtcttgctgaaagaggtaggaggcatgcatctgattgcatgactgcatcaatccctcttcac
gcacaagaagagcagaacattgaagaggaacacccatcatcttgtgtggattccatgtgacagagttggcacgttcaa
caccactcagcttccacttgtgtttccttgacatgagcaaccctccaccccaggcagcatccacatgcatccagatcttgta
cttcttgcagatatcagcaacagccaagagaggatcaaaggcaccgtacacagttgtgccagcagtggctgacaccaa
gaagggaacaaagcctttctgtttggcttcaaggattcgacgttcaagatcagagggaaccatcttgcctctctcatcaca
cttgatgagtatcacactgtctgtgcctatgcccagtgctgcagctcctttcttgaggctgaaatgactgtgttctgatgtga
atgcaatgagcctcggaacagctgccatgcctttctctttgacttctggaaacatcttgaatctggcaatgagcatggcat
acatgttggagatggctccaccaggagagaagatcccatctccagagccaccaggccagccaatgatctccctcatcttc
ttgagtgtcacatactccagcagaacaaagacaggagctatctcataggtgaacatgtttgtgttggcagtggatgtcaa
ccaatcagctgcaagaccaaccatgtccaacccagtggacaactgattgaagtaacgaggatgaccagtcttgatagc
atacttgagagttgtttggcaatgcatcagtatctcttcaagattctgaggttgatctgccaactcccagttgtactcttgga
gaagctcattcggatagtgaaagtcaatcactttggtagatctgtcaaatgacttcacaacatactgcaagagaatgtcc
atgacatcttgaaggaatgccagagtcggacgttctccatcgcaggctggcaacagatcagtggcatgcaggaatgcg
tagttgacttcagctttgggacaactgcagggtttctgattgcaggcacaggcagctttcctactggtagctctgggtggtt
cacttccaccagattctgctggtttctcagcatctccatacaagagagcacaaagtttgttcccaatgccaccagtgaactt
ctgtgcaacttggcaccatgctcgtgcagttgatggattctcagggtccccagatccgtcctcggagccaaaggaccaga
aaccgcttccgggtgatgccatggagatctacaaacttacaaatttctctgaagttgtatcctcagtacttcaaagaaaat
agcttacaccaaattttttcttgttttcacaaatgccgaacttggttccttatataggaaaactcaagggcaaaaatgacac
ggaaaaatataaaaggataagtagtgggggataagattcctttgtgataaggttactttccgcccttacattttccacctt
acatgtgtcctctatgtctctttcacaatcaccgaccttatcttcttcttttcattgttgtcgtcagtgcttacgtcttcaagattc
ttttcttcgcctggttcttctttttcaatttctacgtattcttcttcgtattctggcagtataggatcttgtatctgtacattcttcat
ttttgaacataggttgcatatgtgccgcatattgatctgcttcttgctgagcttacataatacttccatagtttttcccgtaaac
attggattcttgatgctacatcttggataattaccttctgggtttagcggccgcggtgaagggggcggccgcggagcctg
cttttttgtacaaacttgtgcggccgcttgcccgggcatggcgcgccttaattaagcggtggccactattttcagaagaagt
tcccaatagtagtccaaaatttttgtaacgaagggagcataatagttacatgcaaaggaaaactgccattctttagaggg
gatgcttgtttaagaacaaaaaatatatcactttcttttgttccaagtcattgcgtatttttttaaaaatatttgttccttcgtat
atttcgagcttcaatcactttatggttcattgtattctggctttgctgtaaatcgtagctaaccttcttcctagcagaaattatt
aatacttgggatatttttttagaatcaagtaaattacatattaccaccacatcgagctgcttttaaattcatattacagccat
ataggcttgattcattttgcaaaatttccaggatattgacaacgttaacttaataatatcttgaaatattaaagctattatga
ttaggggtgcaaatggaccgagttggttcggtttatatcaaaatcaaaccaaaccaactatatcggtttggattggttcgg
ttttgccgggttttcagcattttctggttttttttttgttagatgaatattattttaatcttactttgtcaaatttttgataagtaaat
atatgtgttagtaaaaattaattttttttacaaacatatgatctattaaaatattcttataggagaattttcttaataacacat
gatatttatttattttagtcgtttgactaatttttcgttgatgtacactttcaaagttaaccaaatttagtaattaagtataaaa
atcaatatgatacctaaataatgatatgttctatttaattttaaattatcgaaatttcacttcaaattcgaaaaagatatata
agaattttgataggttttgacatatgaatatggaagaacaaagagattgacgcattttagtaacacttgataagaaagtg
atcgtacaaccaattatttaaagttaataaaaatggagcacttcatatttaacgaaatattacatgccagaagagtcgca
aatatttctagatattttttaaagaaaattctataaaaagtcttaaaggcatatatataaaaactatatatttatattttggttt
ggttcgaatttgttttactcaataccaaactaaattagaccaaatataattgagatttttaatcgcggcccatgatcacacc
ggttaggatccggtgagtaatattgtacggctaagagcgaatttggcctgtagacctcaattgcgagctttctaatttcaa
actattcgggcctaacttttggtgtgatgatgctgactggcaggatatataccgttgtaatttgagctcgtgtgaataagtc
gctgtgtatgtttgtttgattgtttctgttggagtgcagcccatttcaccggacaagtcggctagattgatttagccctgatg
aactgccgaggggaagccatcttgagcgcggaatgggaatggatcgaaccgggagcacaggatgacgcctaacaat
tcattcaagccgacaccgcttcgcggcgcggcttaattcaggagttaaacatcatgagggaagcggtgatcgccgaagt
atcgactcaactatcagaggtagttggcgtcatcgagcgccatctcgaaccgacgttgctggccgtacatttgtacggctc
cgcagtggatggcggcctgaagccacacagtgatattgatttgctggttacggtgaccgtaaggcttgatgaaacaacg
cggcgagctttgatcaacgaccttttggaaacttcggcttcccctggagagagcgagattctccgcgctgtagaagtcac
cattgttgtgcacgacgacatcattccgtggcgttatccagctaagcgcgaactgcaatttggagaatggcagcgcaatg
acattcttgcaggtatcttcgagccagccacgatcgacattgatctggctatcttgctgacaaaagcaagagaacatagc
gttgccttggtaggtccagcggcggaggaactctttgatccggttcctgaacaggatctatttgaggcgctaaatgaaac
cttaacgctatggaactcgccgcccgactgggctggcgatgagcgaaatgtagtgcttacgttgtcccgcatttggtaca
gcgcagtaaccggcaaaatcgcgccgaaggatgtcgctgccgactgggcaatggagcgcctgccggcccagtatcag
cccgtcatacttgaagctaggcaggcttatcttggacaagaagatcgcttggcctcgcgcgcagatcagttggaagaatt
tgttcactacgtgaaaggcgagatcaccaaggtagtcggcaaataatgtctaacaattcgttcaagccgacgccgcttcg
cggcgcggcttaactcaagcgttagagagctggggaagactatgcgcgatctgttgaaggtggttctaagcctcgtactt
gcgatggcatttcgatcgaaaggggtacaaattcccactaagcgctcgggggctgagaaagcccagtaaggaaacaa
ctgtaggttcgagtcgcgagatcccccggaaccaaaggaagtaggttaaacccgctccgatcaggccgagccacgcca
ggccgagaacattggttcctgtaggcatcgggattggcggatcaaacactaaagctactggaacgagcagaagtcctc
cggccgccagttgccaggcggtaaaggtgagcagaggcacgggaggttgccacttgcgggtcagcacggttccgaac
gccatggaaaccgcccccgccaggcccgctgcgacgccgacaggatctagcgctgcgtttggtgtcaacaccaacagc
gccacgcccgcagttccgcaaatagcccccaggaccgccatcaatcgtatcgggctacctagcagagcggcagagatg
aacacgaccatcagcggctgcacagcgcctaccgtcgccgcgaccccgcccggcaggcggtagaccgaaataaacaa
caagctcca

TABLE D
Sequence ID 4: native canine GAD65

atggcatcacccggaagcggtttctggtcctttggctccgaggacggatctggggaccctgagaatccatcaactgcac
gagcatggtgccaagttgcacagaagttcactggtggcattgggaacaaactttgtgctctcttgtatggagatgctgag
aaaccagcagaatctggtggaagtgaaccacccagagctaccagtaggaaagctgcctgtgcctgcaatcagaaacc
ctgcagttgtcccaaagctgaagtcaactacgcattcctgcatgccactgatctgttgccagcctgcgatggagaacgtcc
gactctggcattccttcaagatgtcatggacattctcttgcagtatgttgtgaagtcatttgacagatctaccaaagtgattg
actttcactatccgaatgagcttctccaagagtacaactgggagttggcagatcaacctcagaatcttgaagagatactg
atgcattgccaaacaactctcaagtatgctatcaagactggtcatcctcgttacttcaatcagttgtccactgggttggaca
tggttggtcttgcagctgattggttgacatccactgccaacacaaacatgttcacctatgagatagctcctgtctttgttctg
ctggagtatgtgacactcaagaagatgagggagatcattggctggcctggtggctctggagatgggatcttctctcctgg
tggagccatctccaacatgtatgccatgctcattgccagattcaagatgtttccagaagtcaaagagaaaggcatggca
gctgttccgaggctcattgcattcacatcagaacacagtcatttcagcctcaagaaaggagctgcagcactgggcatag
gcacagacagtgtgatactcatcaagtgtgatgagagaggcaagatggttccctctgatcttgaacgtcgaatccttgaa
gccaaacagaaaggctttgttcccttcttggtgtcagccactgctggcacaactgtgtacggtgcctttgatcctctcttgg
ctgttgctgatatctgcaagaagtacaagatctggatgcatgtggatgctgcctggggtggagggttgctcatgtcaagg
aaacacaagtggaagctgagtggtgttgaacgtgccaactctgtcacatggaatccacacaagatgatgggtgttcctc
ttcaatgttctgctcttcttgtgcgtgaagagggattgatgcagtcatgcaatcagatgcatgcctcctacctctttcagcaa
gacaaacactatgatctgtcctatgacactggagacaaagccttgcagtgtggacgacatgttgatgtcttcaagctctg
gctcatgtggagagccaaagggaccactgggtttgaggctcacatagacaagtgtttggagcttgcagagtacctgtac
agcattatcaagaatcgagaaggctacgagatggtgtttgatgggaagccacaacacaccaatgtctgcttctggtatgt
tcctccgtcattgagggttctggaagacaacgaagagaggatgaacagactctcaaaggtggcaccagtgatcaaggc
aaggatgatggagtatgggaccacaatggtcagctaccagccactgggagacaaggtgaacttctttcgaatggtcatc
tccaatcctgctgcaactcatcaagacattgactttctcattgaagagattgaaagacttggacaagatctt

TABLE E

Sequence ID 5: canine GAD65 optimized for plant
expression

atggcatcacccggaagcggtttctggtcctttggctccgaggacggatctggggaccctgagaatccatcaactgcac
gagcatggtgccaagttgcacagaagttcactggtggcattgggaacaaactttgtgctctcttgtatggagatgctgag
aaaccagcagaatctggtggaagtgaaccacccagagctaccagtaggaaagctgcctgtgcctgcaatcagaaacc
ctgcagttgtcccaaagctgaagtcaactacgcattcctgcatgccactgatctgttgccagcctgcgatggagaacgtcc
gactctggcattccttcaagatgtcatggacattctcttgcagtatgttgtgaagtcatttgacagatctaccaaagtgattg
actttcactatccgaatgagcttctccaagagtacaactgggagttggcagatcaacctcagaatcttgaagagatactg
atgcattgccaaacaactctcaagtatgctatcaagactggtcatcctcgttacttcaatcagttgtccactgggttggaca
tggttggtcttgcagctgattggttgacatccactgccaacacaaacatgttcacctatgagatagctcctgtctttgttctg
ctggagtatgtgacactcaagaagatgagggagatcattggctggcctggtggctctggagatgggatcttctctcctgg
tggagccatctccaacatgtatgccatgctcattgccagattcaagatgtttccagaagtcaaagagaaaggcatggca
gctgttccgaggctcattgcattcacatcagaacacagtcatttcagcctcaagaaaggagctgcagcactgggcatag
gcacagacagtgtgatactcatcaagtgtgatgagagaggcaagatggttccctctgatcttgaacgtcgaatccttgaa
gccaaacagaaaggctttgttcccttcttggtgtcagccactgctggcacaactgtgtacggtgcctttgatcctctcttgg
ctgttgctgatatctgcaagaagtacaagatctggatgcatgtggatgctgcctggggtggagggttgctcatgtcaagg
aaacacaagtggaagctgagtggtgttgaacgtgccaactctgtcacatggaatccacacaagatgatgggtgttcctc
ttcaatgttctgctcttcttgtgcgtgaagagggattgatgcagtcatgcaatcagatgcatgcctcctacctctttcagcaa
gacaaacactatgatctgtcctatgacactggagacaaagccttgcagtgtggacgacatgttgatgtcttcaagctctg
gctcatgtggagagccaaagggaccactgggtttgaggctcacatagacaagtgtttggagcttgcagagtacctgtac
agcattatcaagaatcgagaaggctacgagatggtgtttgatgggaagccacaacacaccaatgtctgcttctggtatgt
tcctccgtcattgagggttctggaagacaacgaagagaggatgaacagactctcaaaggtggcaccagtgatcaaggc
aaggatgatggagtatgggaccacaatggtcagctaccagccactgggagacaaggtgaacttctttcgaatggtcatc
tccaatcctgctgcaactcatcaagacattgactttctcattgaagagattgaaagacttggacaagatctt

TABLE F
Sequence ID 6: pDAB2453

ggccgcttaattaaatttaaatgtttaaactaggaaatccaagcttgggctgcaggtcaatcccattgcttttgaagcagct
caacattgatctctttctcgaggtcattcatatgcttgagaagagagtcgggatagtccaaaataaaacaaaggtaagat
tacctggtcaaaagtgaaaacatcagttaaaaggtggtataaagtaaaatatcggtaataaaaggtggcccaaagtga
aatttactcttttctactattataaaaattgaggatgtttttgtcggtactttgatacgtcatttttgtatgaattggtttttaagt
ttattcgcttttggaaatgcatatctgtatttgagtcgggttttaagttcgtttgcttttgtaaatacagagggatttgtataa
gaaatatctttaaaaaaacccatatgctaatttgacataatttttgagaaaaatatatattcaggcgaattctcacaatga
acaataataagattaaaatagctttcccccgttgcagcgcatgggtattttttctagtaaaaataaaagataaacttagac
tcaaaacatttacaaaaacaacccctaaagttcctaaagcccaaagtgctatccacgatccatagcaagcccagcccaa
cccaacccaacccaacccaccccagtccagccaactggacaatagtctccacacccccccactatcaccgtgagttgtcc
gcacgcaccgcacgtctcgcagccaaaaaaaaaaaaagaaagaaaaaaaagaaaaagaaaaaacagcaggtggg
tccgggtcgtgggggccggaaacgcgaggaggatcgcgagccagcgacgaggccggccctccctccgcttccaaaga
aacgccccccatcgccactatatacatacccccccctctcctcccatccccccaaccctaccaccaccaccaccaccacctc
cacctcctcccccctcgctgccggacgacgcctcccccctccccctccgccgccgccgcgccggtaaccaccccgcccctc
tcctctttctttctccgttttttttttccgtctcggtctcgatctttggccttggtagtttgggtgggcgagaggcggcttcgtgc
gcgcccagatcggtgcgcgggaggggcgggatctcgcggctggggctctcgccggcgtggatccggcccggatctcg
cggggaatggggctctcggatgtagatctgcgatccgccgttgttgggggagatgatggggggtttaaaatttccgcca
tgctaaacaagatcaggaagaggggaaaagggcactatggtttatatttttatatatttctgctgcttcgtcaggcttaga
tgtgctagatctttctttcttctttttgtgggtagaatttgaatccctcagcattgttcatcggtagtttttcttttcatgatttgtg
acaaatgcagcctcgtgcggagcttttttgtaggtagaccatggcttctccggagaggagaccagttgagattaggcca
gctacagcagctgatatggccgcggtttgtgatatcgttaaccattacattgagacgtctacagtgaactttaggacaga
gccacaaacaccacaagagtggattgatgatctagagaggttgcaagatagatacccttggttggttgctgaggttgag
ggtgttgtggctggtattgcttacgctgggccctggaaggctaggaacgcttacgattggacagttgagagtactgttta
cgtgtcacataggcatcaaaggttgggcctaggatccacattgtacacacatttgcttaagtctatggaggcgcaaggtt
ttaagtctgtggttgctgttataggccttccaaacgatccatctgttaggttgcatgaggctttgggatacacagcccggg
gtacattgcgcgcagctggatacaagcatggtggatggcatgatgttggtttttggcaaagggattttgagttgccagct
cctccaaggccagttaggccagttacccagatctgaggtaccctgagctcggtcgcagcgtgtgcgtgtccgtcgtacgt
tctggccggccgggccttgggcgcgcgatcagaagcgttgcgttggcgtgtgtgtgcttctggtttgctttaattttaccaa
gtttgtttcaaggtggatcgcgtggtcaaggcccgtgtgctttaaagacccaccggcactggcagtgagtgttgctgcttg
tgtaggctttggtacgtatgggctttatttgcttctggatgttgtgtactacttgggtttgttgaattattatgagcagttgcgt
attgtaattcagctgggctacctggacattgttatgtattaataaatgctttgctttcttctaaagatctttaagtgctgaattc
atatttcctcctgcagggtttaaacttgccgtggcctattttcagaagaagttcccaatagtagtccaaaatttttgtaacga
agggagcataatagttacatgcaaaggaaaactgccattctttagaggggatgcttgtttaagaacaaaaaatatatca
ctttcttttgttccaagtcattgcgtatttttttaaaaatatttgttccttcgtatatttcgagcttcaatcactttatggttctttgt
attctggctttgctgtaaatcgtagctaaccttcttcctagcagaaattattaatacttgggatatttttttagaatcaagtaa
attacatattaccaccacatcgagctgcttttaaattcatattacagccatataggcttgattcattttgcaaaatttccagg
atattgacaacgttaacttaataatatcttgaaatattaaagctattatgattaggggtgcaaatggaccgagttggttcg
gtttatatcaaaatcaaaccaaaccaactatatcggtttggattggttcggttttgccgggttttcagcattttctggttttttt
tttgttagatgaatattattttaatcttactttgtcaaatttttgataagtaaatatatgtgttagtaaaaattaattttttttaca
aacatatgatctattaaaatattcttataggagaattttcttaataacacatgatatttatttattttagtcgtttgactaatttt
tcgttgatgtacactttcaaagttaaccaaatttagtaattaagtataaaaatcaatatgatacctaaataatgatatgttct
atttaattttaaattatcgaaatttcacttcaaattcgaaaaagatatataagaattttgatagattttgacatatgaatatg
gaagaacaaagagattgacgcattttagtaacacttgataagaaagtgatcgtacaaccaattatttaaagttaataaa
aatggagcacttcatatttaacgaaatattacatgccagaagagtcgcaaatatttctagatattttttaaagaaaattcta
taaaaagtcttaaaggcatatatataaaaactatatatttatattttggtttggttcgaatttgttttactcaataccaaacta
aattagaccaaatataattgggatttttaatcgcggcccactagtcaccggtgtagcttggcgtaatcatggtcatagctg
tttcctgtgtgaaattgttatccgctcacaattccacacaacatacgagccggaagcataaagtgtaaagcctggggtgc
ctaatgagtgagctaactcacattaattgcgttgcgctcactgcccgctttccagtcgggaaacctgtcgtgccagctgca
ttaatgaatcggccaacgcgcggggagaggcggtttgcgtattgggcgctcttccgctgcgcacgctgcgcacgctgcg
cacgcttcctcgctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggccaggaaccg
taaaaaggccgcgttgctggcgtttttccataggctccgcccccctgacgagcatcacaaaaatcgacgctcaagtcaga
ggtggcgaaacccgacaggactataaagataccaggcgtttccccctggaagctccctcgtgcgctctcctgttccgacc
ctgccgcttaccggatacctgtccgcctttctcccttcgggaagcgtggcgctttctcatagctcacgctgtaggtatctcag
ttcggtgtaggtcgttcgctccaagctgggctgtgtgcacgaaccccccgttcagcccgaccgctgcgccttatccggtaa
ctatcgtcttgagtccaacccggtaagacacgacttatcgccactggcagcagccactggtaacaggattagcagagcg
aggtatgtaggcggtgctacagagttcttgaagtggtggcctaactacggctacactagaaggacagtatttggtatctg
cgctctgctgaagccagttaccttcggaaaaagagttggtagctcttgatccggcaaacaaaccaccgctggtagcggt
ggtttttttgtttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcacctagatcctttta
aattaaaaatgaagttttaaatcaatctaaagtatatatgagtaaacttggtctgacagttaccaatgcttaatcagtgag
gcacctatctcagcgatctgtctatttcgttcatccatagttgcctgactccccgtcgtgtagataactacgatacgggagg
gcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagc
tagagtaagtagttcgccagttaatagtttgcgcaacgttgttgccattgctacaggcatcgtggtgtcacgctcgtcgttt
ggtatggcttcattcagctccggttcccaacgatcaaggcgagttacatgatcccccatgttgtgcaaaaaagcggttag
ctccttcggtcctccgatcgttgtcagaagtaagttggccgcagtgttatcactcatggttatggcagcactgcataattctc
ttactgtcatgccatccgtaagatgcttttctgtgactggtgagtactcaaccaagtcattctgagaatagtgtatgcggcg
accgagttgctcttgcccggcgtcaatacgggataataccgcgccacatagcagaactttaaaagtgctcatcattggaa
aacgttcttcggggcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgcaaaaaagggaat
aagggcgacacggaaatgttgaatactcatactcttcctttttcaatattattgaagcatttatcagggttattgtctcatga
gcggatacatatttgaatgtatttagaaaaataaacaaataggggttccgcgcacatttccccgaaaagtgccacctgac
gtctaagaaaccattattatcatgacattaacctataaaaataggcgtatcacgaggccctttcgtctcgcgcgtttcggtg
atgacggtgaaaacctctgacacatgcagctcccggagacggtcacagcttgtctgtaagcggatgccgggagcagac
aagcccgtcagggcgcgtcagcgggtgttggcgggtgtcggggctggcttaactatgcggcatcagagcagattgtact
gagagtgcaccatatgcggtgtgaaataccgcacagatgcgtaaggagaaaataccgcatcaggcgccattcgccatt
caggctgcgcaactgttgggaagggcgatcggtgcgggcctcttcgctattacgccagctggcgaaagggggatgtgc
tgcaaggcgattaagttgggtaacgccagggttttcccagtcacgacgttgtaaaacgacggccagtgaattacaccgg
tgtgatcatgggccgcgattaaaaatctcaattatatttggtctaatttagtttggtattgagtaaaacaaattcgaaccaa
accaaaatataaatatatagtttttatatatatgcctttaagactttttatagaattttctttaaaaaatatctagaaatatttg
cgactcttctggcatgtaatatttcgttaaatatgaagtgctccatttttattaactttaaataattggttgtacgatcactttct
tatcaagtgttactaaaatgcgtcaatctctttgttcttccatattcatatgtcaaaacctatcaaaattcttatatatctttttc
gaatttgaagtgaaatttcgataatttaaaattaaatagaacatatcattatttaggtatcatattgatttttatacttaatta
ctaaatttggttaactttgaaagtgtacatcaacgaaaaattagtcaaacgactaaaataaataaatatcatgtgttatta
agaaaattctcctataagaatattttaatagatcatatgtttgtaaaaaaaattaatttttactaacacatatatttacttatc
aaaaatttgacaaagtaagattaaaataatattcatctaacaaaaaaaaaaccagaaaatgctgaaaacccggcaaa
accgaaccaatccaaaccgatatagttggtttggtttgattttgatataaaccgaaccaactcggtccatttgcacccctaa
tcataatagctttaatatttcaagatattattaagttaacgttgtcaatatcctggaaattttgcaaaatgaatcaagcctat
atggctgtaatatgaatttaaaagcagctcgatgtggtggtaatatgtaatttacttgattctaaaaaaatatcccaagtat
taataatttctgctaggaagaaggttagctacgatttacagcaaagccagaatacaatgaaccataaagtgattgaagc
tcgaaatatacgaaggaacaaatatttttaaaaaaatacgcaatgacttggaacaaaagaaagtgatatattttttgttct
taaacaagcatcccctctaaagaatggcagttttcctttgcatgtaactattatgctcccttcgttacaaaaattttggacta
ctattgggaacttcttctgaaaatagtggccaccgcttaattaaggcgcgccatgcccgggcaagcggccgcattcccgg
gaagctaggccaccgtggcccgcctgcaggggaagcttgcatgcctgcagatccccggggatcctctagagtcgacct
gcagtgcagcgtgacccggtcgtgcccctctctagagataatgagcattgcatgtctaagttataaaaaattaccacatat
tttttttgtcacacttgtttgaagtgcagtttatctatctttatacatatatttaaactttactctacgaataatataatctatagt
actacaataatatcagtgttttagagaatcatataaatgaacagttagacatggtctaaaggacaattgagtattttgaca
acaggactctacagttttatctttttagtgtgcatgtgttctcctttttttttgcaaatagcttcacctatataatacttcatccat
tttattagtacatccatttagggtttagggttaatggtttttatagactaatttttttagtacatctattttattctattttagcctc
taaattaagaaaactaaaactctattttagtttttttatttaatagtttagatataaaatagaataaaataaagtgactaaa
aattaaacaaataccctttaagaaattaaaaaaactaaggaaacatttttcttgtttcgagtagataatgccagcctgtta
aacgccgtcgacgagtctaacggacaccaaccagcgaaccagcagcgtcgcgtcgggccaagcgaagcagacggca
cggcatctctgtcgctgcctctggacccctctcgagagttccgctccaccgttggacttgctccgctgtcggcatccagaaa
ttgcgtggcggagcggcagacgtgagccggcacggcaggcggcctcctcctcctctcacggcaccggcagctacggg
ggattcctttcccaccgctccttcgctttcccttcctcgcccgccgtaataaatagacaccccctccacaccctctttccccaa
cctcgtgttgttcggagcgcacacacacacaaccagatctcccccaaatccacccgtcggcacctccgcttcaaggtacg
ccgctcgtcctccccccccccccccctctctaccttctctagatcggcgttccggtccatgcatggttagggcccggtagttc
tacttctgttcatgtttgtgttagatccgtgtttgtgttagatccgtgctgctagcgttcgtacacggatgcgacctgtacgtc
agacacgttctgattgctaacttgccagtgtttctctttggggaatcctgggatggctctagccgttccgcagacgggatc
gatttcatgattttttttgtttcgttgcatagggtttggtttgcccttttcctttatttcaatatatgccgtgcacttgtttgtcggg
tcatcttttcatgcttttttttgtcttggttgtgatgatgtggtctggttgggcggtcgttctagatcggagtagaattctgtttc
aaactacctggtggatttattaattttggatctgtatgtgtgtgccatacatattcatagttacgaattgaagatgatggatg
gaaatatcgatctaggataggtatacatgttgatgcgggttttactgatgcatatacagagatgctttttgttcgcttggttg
tgatgatgtggtgtggttgggcggtcgttcattcgttctagatcggagtagaatactgtttcaaactacctggtgtatttatt
aattttggaactgtatgtgtgtgtcatacatcttcatagttacgagtttaagatggatggaaatatcgatctaggataggta
tacatgttgatgtgggttttactgatgcatatacatgatggcatatgcagcatctattcatatgctctaaccttgagtaccta
tctattataataaacaagtatgttttataattatttcgatcttgatatacttggatgatggcatatgcagcagctatatgtgg
atttttttagccctgccttcatacgctatttatttgcttggtactgtttcttttgtcgatgctcaccctgttgtttggtgttacttct
gcagggtacccccggggtcgaccatgggcctcacatcacaactgattccgactcttgtctgtctccttgccctcacctccac
atttgttcatggacacaacttcaatatcaccattaaggagataatcaaaatgttgaacattttgacagcaaggaatgata
gttgcatggagctgactgtgaaggatgttttcactgctcctaagaacacttcagacaaagagattttctgccgtgctgcca
ctgtcctcagacaaatctacacccacaactgctccaatagatacttgaggggtctttatcgaaatctcagctcaatggcaa
acaaaacctgtagcatgaatgaaatcaagaaatctacattgaaagactttctggaaaggctgaaagtgataatgcaga
agaagtattacagacatcatcaccatcaccattgagtagttagcttaatcacctagagctcgtttaaactgagggcactg
aagtcgcttgatgtgctgaattgtttgtgatgttggtggcgtattttgtttaaataagtaagcatggctgtgattttatcatat
gatcgatctttggggttttatttaacacattgtaaaatgtgtatctattaataactcaatgtataagatgtgttcattcttcggt
tgccatagatctgcttatttgacctgtgatgttttgactccaaaaaccaaaatcacaactcaataaactcatggaatatgtc
cacctgtttcttgaagagttcatctaccattccagttggcatttatcagtgttgcagcggcgctgtgctttgtaacataacaa
ttgttacggcatatatccaacggccggcctaggccacggtggccagatccactagttctagagc

TABLE G

Sequence ID 7: canine IL-4 optimized for plant
expression with a 6 histidine tag

atgggcctcacatcacaactgattccgactcttgtctgtctccttgccctcacctccacatttgttcatggacacaacttcaat
atcaccattaaggagataatcaaaatgttgaacattttgacagcaaggaatgatagttgcatggagctgactgtgaagg
atgttttcactgctcctaagaacacttcagacaaagagattttctgccgtgctgccactgtcctcagacaaatctacaccca
caactgctccaatagatacttgaggggtctttatcgaaatctcagctcaatggcaaacaaaacctgtagcatgaatgaaa
tcaagaaatctacattgaaagactttctggaaaggctgaaagtgataatgcagaagaagtattacagacatcatcacca
tcaccat

Although preferred embodiments of the invention have been described herein in detail, it will be understood by those skilled in the art that variations may be made thereto without departing from the spirit of the invention or the scope of the appended claims.

1. A method for the treatment of new onset Type I diabetes in a mammal or pre-Type I diabetic mammal, the method comprising;
(a) administering anti-T cell therapy to said mammal; and
(b) administering an autoantigen composition comprising an optional mucosal antigen, wherein (a) and (b) are administered concurrently or sequentially.

2. The method of claim 1, wherein said anti-T cell therapy is an immunosuppressant agent that targets T cells.

3. The method of claim 2, wherein said immunosuppressant agent is selected from the group consisting of monoclonal antibodies targeting T cell surface antigens, polyclonal antibodies targeting T cell surface antigens, cyclosporine, methotrexate, azathioprine and combinations thereof.

4. The method of claim 3, wherein said immunosuppressant agent is a monoclonal antibody selected from the group consisting of anti CD3, anti CD2, anti CD4 anti CD7, anti CD8, anti CD25, anti CD28, anti alpha 4 beta 1 integrin, anti alpha 4 beta 7 integrin and combinations thereof.

5. The method of claim 4, wherein said immunosuppressant is an anti CD3 monoclonal antibody.

6. The method of claim 1, wherein said autoantigen is selected from the group consisting of a GAD isoform, GAD polypeptide, insulin and combinations thereof.

7. The method of claim 6, wherein said autoantigen is a GAD isoform selected from the group consisting of GAD65, GAD67 and mixtures thereof.

8. The method of claim 1, wherein said mucosal antigen is an immunoregulatory cytokine.

9. The method of claim 8, wherein said immunoregulatory cytokine is an interleukin.

10. The method of claim 9, wherein said interleukin is selected from the group consisting of IL-1, IL2, IL-3, IL4, IL5, IL6, IL-7, IL-8, IL9, IL-10, IL-12, IL-13, IL15, IL18 and mixtures thereof.

11. The method of claim 10, wherein said interleukin is IL-4.

12. The method of claim 10, wherein said interleukin is IL10.

13. The method of claim 2, wherein said immunosuppressant agent is administered intravenously to said mammal for up to about 10 days.

14. The method of claim 13, wherein immunosuppressant agent is administered intravenously to said mammal for up to about 5 to 7 days.

15. The method of claim 13, wherein said immunosuppressant agent is administered at dosages of up to about 10μ/kg to up to about 100μ/kg body weight.

16. The method of claim 1, wherein said autoantigen and optional mucosal antigen composition is administered orally or to a mucosal surface or parentally.

17. The method of claim 16, wherein said autoantigen and optional mucosal antigen composition is provided within a transgenic plant material.

18. The method of claim 17, wherein said transgenic plant material is selected from the group consisting of potato, tomato, alfalfa, canola, rice, tobacco, maize, algae, safflower, moss and bryophyte.

19. The method of claim 17, wherein said transgenic plant material is selected from the group consisting of plant tissue, plant leaves, plant tubers, plant stems, plant extracts, plant slurries, plant cell cultures and combinations thereof.

20. The method of claim 17, wherein said composition s administered orally.

21. The method of claim 16, wherein said autoantigen and optional mucosal antigen is provided in an amount of up to about 1 mg/kg to up to about 1000 mg/kg.

22. The method of claim 16, wherein said autoantigen and optional mucosal antigen is provided in an amount of more than about 1000 mg/kg.

23. The method of claim 21, wherein said autoantigen and optional mucosal antigen is provided in an amount of up to about 1 mg/kg to up to about 100 mg/kg.

24. The method of claim 1, wherein (a) and (b) are administered concurrently.

25. The method of claim 1, wherein (a) and (b) are administered sequentially.

26. The method of claim 1, wherein (a) and (b) are administered concurrently followed by further administration of (b).

27. The method of claim 26, wherein said further administration of (b) is done for an extended period of time.

28. The method of claim 27, wherein said extended period of time is up to about the lifespan of the mammal.

29. The method of claim 1, wherein said mammal is a human.

30. The method of claim 1, wherein said mammal is a companion animal selected from the group consisting of dogs, cats and horses.

31. The method of claim 29, wherein said human has new onset Type I diabetes.

32. The method of claim 29, wherein said human s pre-Type I diabetic.

33. A method for treating Type I diabetes in a mammal or for treating pre-type I diabetic mammals, the method comprising:
(a) administering an effective dose of anti-T cell antibodies to said human: and
(b) administering an effective dose of an autoantigen to said mammal, wherein (a) and (b) are administered at the same time or sequentially for an effective time period, or (a) and (b) are administered at the same time and (b) is further administered alone for a longer time period.

34. A method for treating Type I diabetes in a human, or for treating pre-type I diabetic humans, the method comprising;
(a) administering an effective immunosuppressive dose of anti-T cel antibodies to said humans; and
(b) administering an effective immunosuppressive dose of a transgenic plant material to said mammal, said transgenic plant material containing at least one autoantigen and optionally at least one immunoregulatory cytokine;
wherein said administering of (a) and (b) is done concurrently or sequentially.

35. The method of claim 34, wherein said anti-T cell antibodies are polyclonal antibodies.

36. The method of claim 34, wherein said anti-T cell antibodies are monoclonal antibodies.

37. The method of claim 36, wherein said monoclonal antibody is selected from the group consisting of anti CD3, anti CD2, anti CD4, anti CD7, anti CD8, anti CD25 anti CD28, anti alpha 4 beta 1 integrin, anti alpha 4 beta 7 integrin and combinations thereof.

38. The method of claim 37, wherein said monoclonal antibody is an anti CD3 monoclonal antibody.

39. The method of claim 34, wherein said autoantigen is selected from the group consisting of GAD isoform, GAD polypeptide, insulin and combinations thereof.

40. The method of claim 39, wherein said autoantigen is a GAD isoform selected from the group consisting of GAD65, GAD67 and mixtures thereof.

41. The method of claim 34, wherein said immunoregulatory cytokine is an interleukine.

42. The method of claim 41, wherein said interleukin is selected from the group consisting of IL-1, IL-2, IL-3, IL-4, IL-5, IL-6, IL-7, IL-8, IL-9, IL-10, IL-12, IL-13, IL-15, IL-18 and mixtures thereof.

43. The method of claim 42, wherein said interleukin is IL-4.

44. The method of claim 42, wherein said interleukin is IL-10.

45. The method of claim 41, wherein said transgenic plant maternal is administered orally or to a mucosal surface.

46. The method of claim 45, wherein said transgenic plant material is administered to provide up to about 10 μg/kg to up to about 100 μg/kg body weight of said anti-T cell antibodies.

47. The method of claim 46, wherein said transgenic plant material provides about up to about 1 mg/kg to up to about 1000 mg/kg of autoantigen and optional immunoregulatory cytokine.

48. The method of claim 34, wherein (a) and (b) are administered concurrently.

49. The method of claim 34, wherein (a) and (b) are administered sequentially.

50. The method of claim 34, wherein (a) and (b) are administered concurrently followed by further administration of (b).

51. The method of claim 50, wherein said further administration of (b) is done for an extended period of time.

52. The method of claim 51, wherein said extended period of time is up to about the lifespan of the mammal.

53. A method for reversal of Type I diabetes in a human or companion animal, said method comprising:
(a) administering a therapeutically effective amount of anti-CD3 monoclonal antibody to said human or animal; and
(b) administering a therapeutically effective amount of a transgenic plant material containing one or more GAD autoantigens together with IL-4,
wherein (a) is first administered to said human or animal.

54. The method of claim 53, wherein (a) and (b) are administered concurrently.

55. The method of claim 53 wherein (b) is further administered for an extended period of time.

56. An IL-4 nucleotide sequence optimized for plant expression.

57. The sequence of claim 56, wherein said optimization is the addition of a histidine tag.

58. The sequence of claim 56, wherein said optimization is the addition of an ER retention signal and histidine tag.

59. The sequence of claim 56, wherein said nucleotide sequence is selected from the group consisting of SEQ ID NO. 2 and SEQ ID NO. 7.

60. The sequence of claim 56, wherein said sequence is a canine sequence.

61. A canine GAD65 nucleotide sequence of SEQ ID NO. 4.

62. The nucleotide sequence of claim 61 wherein said sequence is further optimized for plant expression.

63. The nucleotide sequence of claim 62, wherein said optimized sequence is represented by SEQ ID NO. 5.

64. A vector for cell transformation, said vector selected from the group consisting of SEQ ID NO. 1, SEQ ID NO. 3 and SEQ ID NO. 6.

65. A vector selected from the group consisting of pDAB771; pDAB773; pDAB24O7; pDAB2457; pDAB2455; pDAB2456; pDAB3736; pDAB3741; pDAB3731; pDAB3748; pDAB2453; PDAB4005; pDAB2451; and pDAB85O4.

66. A composition comprising a mixture of anti-CD3 antibodies and a preparation that contains at least one autoantigen and an immunoregulatory cytokine.

67. A composition comprising a mixture of anti-CD3 antibodies and a transgenic plant material that contains at: least one autoantigen and an immunoregulatory cytokine.

68. The use of a composition comprising anti-T cell antibodies, autoantigen and optional mucosal antigen in the manufacture of a medicament for the treatment of Type I diabetes in a mammal.

69. A method for the diagnosis of Type I diabetes in a mammal, the method comprising detecting in a sample from said mammal the presence of anti-GAD antibodies, such detection being an early indicator of the development or the risk of development of Type I diabetes in the mammal.

70. The method of claim 69, wherein the anti-GAD antibodies are canine antibodies.
